(19) United States
(12) Patent Application Publication
(10) Pub. No.: US 2007/0134322 A1
(43) Pub. Date: Jun. 14, 2007

(54) MODIFIED AND PULSATILE RELEASE PHARMACEUTICAL FORMULATIONS OF ESCITALOPRAM

(75) Inventors: Mahendra G. Dedhiya, Pomona, NY (US); Anil Chhettry, Holtsville, NY (US); Yan Yang, Roslyn Heights, NY (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/610,194

(22) Filed: Dec. 13, 2006

Related U.S. Application Data
(60) Provisional application No. 60/750,841, filed on Dec. 14, 2005.

(51) Int. Cl. A61K 9/20; A61K 31/343

(52) U.S. Cl. 424/464; 514/469

(57) ABSTRACT

The present invention relates to modified and pulsatile release pharmaceutical formulations of escitalopram and their use for the treatment of central nervous system disorders, including mood disorders (e.g., major depressive disorder) and anxiety disorders (e.g., generalized anxiety disorder, social anxiety disorder, post traumatic stress disorder, and panic disorder, including panic attacks).

### CROSS-REFERENCE TO RELATED APPLICATIONS

This application claims priority under 35 U.S.C. § 119, to U.S. Provisional Application Ser. No. 60/750,841 filed Dec. 14, 2005, the disclosure of which is hereby incorporated by reference in its entirety.

### FIELD OF THE INVENTION

The present invention relates to modified and pulsatile release pharmaceutical formulations of escitalopram and their use for the treatment of central nervous system (CNS) disorders, including mood disorders (e.g., major depressive disorder) and anxiety disorders (e.g., generalized anxiety disorder, social anxiety disorder, post traumatic stress disorder, obsessive compulsive disorder and panic disorder).

### BACKGROUND OF THE INVENTION

Selective serotonin reuptake inhibitors (hereinafter called SSRIs), such as racemic citalopram and escitalopram, have become first-choice therapeutics in the treatment of depression primarily due to superior efficacy compared to tricyclic antidepressants and monoamine oxidase inhibitors (MAOIs). SSRIs function by inhibiting the reuptake of the neurotransmitter serotonin (5-hydroxytryptamine, 5-HT) by nerve cells at synapses. As a result, serotonin persists in the synaptic gap and has the chance to stimulate receptors of recipient cells.

Escitalopram is the S-enantiomer of citalopram and has the following structure:

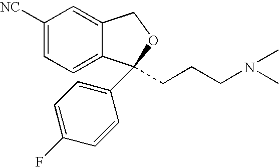

Methods of preparing escitalopram are disclosed in, for example, U.S. Pat. Nos. Re. 34,712 and 6,566,540 and International Publication Nos. WO 03/000672, WO 03/006449, WO 03/051861, and WO 2004/083197.

International Publication Nos. WO 01/03694 and WO 02/087566 disclose the use of escitalopram in the treatment of various mental disorders including major depressive disorder, generalized anxiety disorder, social anxiety disorder, post traumatic stress disorder, panic attacks, acute stress disorder, eating disorders (such as bulimia, anorexia, and obesity), phobias, dysthymia, premenstrual syndrome, cognitive disorders, impulse control disorders, attention deficit hyperactivity disorder, and drug abuse. International Publication No. WO 02/087566 also discloses the use of escitalopram for the treatment of patients who have failed to respond to initial treatment with a conventional SSRI, in particular patients with major depressive disorder who have failed to respond to initial treatment with a conventional SSRI.

Escitalopram oxalate is currently marketed in the United States as Lexapro® for the treatment of major depressive disorder and generalized anxiety disorder. Lexapro® is available in 5, 10, and 20 mg escitalopram immediate release (IR) tablets (as an oxalate salt) and as solutions. Escitalopram has also been studied at the 15 mg strength.

Side effects associated with escitalopram include nausea, insomnia, somnolence, increased sweating, fatigue, and sexual dysfunction (including ejaculation disorder, anorgasmia, and decreased libido).

Currently, a dosing regimen of escitalopram of once a day is employed using immediate release tablets. Waugh and Goa, CNS Drugs, 2003, 17(5):343-362, have reported that peak plasma concentrations of immediate release escitalopram are reached in 4-5 hours (80% absolute bioavailability with 56% binding).

In DeVane, J. Clin. Psychiatry 2003, 64 (suppl. 18):14-19, the results of clinical studies of immediate release and controlled release formulations of antidepressants were compared in relation to nausea leading to drug discontinuation. The author noted that while “more stable pharmacokinetic profiles might be the cause for the low occurrence of nausea with some controlled-release newer antidepressants,” a “connection has not been proven.”

Immediate release solid dosage forms permit the release of most, or all, of the active ingredient over a short period of time and make rapid absorption of the drug possible. The rapid absorption of the drug (i.e., a short Tₘₐₓ) may in some cases result in undesirable adverse events. Modified release (MR) solid oral dosage forms permit the release of the active ingredient over an extended period of time to maintain therapeutically effective plasma levels and may also enhance the other pharmacokinetic properties of the active ingredient.

A modified release formulation of escitalopram oxalate prepared by melt granulation is disclosed in International Publication No. WO01/22941, the entire disclosure of which is incorporated herein by reference. The melt granulated composition is substantially homogeneous and contains one or more hydrophilic cellulose ether polymers, a hydrophilic melt binder, and a therapeutically active medicament.

International Publication No. WO 2004/058229 discloses once a day modified release formulations of SSRIs, such as citalopram hydrobromide, and extrapolates the findings to escitalopram oxalate. The modified release formulations, however, have bioequivalent PK profiles (e.g., essentially the same Cₘₐₓ) as the immediate release SSRI dosage forms.

U.S. Patent Publication No. 2005/020838 discloses a controlled release solid dosage form comprising citalopram or escitalopram.

There remains a need for modified release formulations of escitalopram with enhanced pharmacokinetic properties. Accordingly, the present invention provides modified release dosage forms containing escitalopram that exhibit an enhanced release profile over a targeted period of time and provide fewer Cₘₐₓ-related adverse events.

### SUMMARY OF THE INVENTION

The present invention relates to modified and pulsatile release pharmaceutical formulations of escitalopram and their use for the treatment of central nervous system (CNS) disorders, including mood disorders (e.g., major depressive disorder) and anxiety disorders (e.g., generalized anxiety disorder, social anxiety disorder, post traumatic stress disorder, obsessive compulsive disorder and panic disorder). In particular, the present invention provides modified release oral dosage forms of escitalopram or pharmaceutically acceptable salts thereof (preferably escitalopram oxalate) with enhanced pharmacokinetic properties. Thus, the oral dosage forms may provide improved effectiveness in the treatment of central nervous system disorders and fewer side effects than prior escitalopram formulations.

According to some embodiments, the present invention provides oral dosage forms that includes from about 2 mg to about 30 mg escitalopram or a pharmaceutically acceptable salt thereof, wherein the dosage form provides an in vivo plasma profile comprising a mean Tmax of more than about 6 hours, a mean Cmax of less than about 30 ng/ml and a mean AUC_{0-∞} of more than about 60 ng h/ml.

According to other embodiments, the present invention provides oral dosage forms that includes from about 2 to 30 mg escitalopram or a pharmaceutically acceptable salt thereof wherein the dosage form has a dissolution rate of the active ingredient of about 70% to about 80% within about 4 hours to about 24 hours and provides an in vivo plasma profile comprising a mean Cₘₐₓ of less than about 30 ng/ml.

According to other embodiments, the present invention provides oral dosage forms that includes a plurality of beads, each bead includes a core having a diameter from about 1 μm to about 1000 μm, an active ingredient comprising about 2 to about 30 mg escitalopram or a pharmaceutically acceptable salt thereof and a modified release coating, wherein the oral dosage form has a dissolution rate of the active ingredient of about 70% to about 80% within about 4 hours to about 24 hours and wherein the dosage form provides an in vivo plasma profile comprising a mean Cₘₐₓ of less than about 30 ng/ml.

In yet other embodiments, the present invention provides composite dosage forms that include an immediate release component and a modified release component, wherein the immediate release component comprises a first active ingredient comprising about 2 to about 30 mg escitalopram or a pharmaceutically acceptable salt thereof, wherein about 80% of the first active ingredient dissolves within about the first 4 hours following entry of the dosage form into a use environment and wherein the modified release component comprises a second active ingredient comprising about 2 to about 20 mg escitalopram or a pharmaceutically acceptable salt thereof, wherein about 70% to about 80% of the second active ingredient dissolves within about 4 hours to about 24 hours following entry of the dosage form into the use environment.

In still other embodiments, the present invention provides oral dosage forms that include a plurality of beads, each bead comprising a first drug component comprising a core comprising about 500 to about 800 mg sugar per gram of bead, about 30 mg to about 300 mg escitalopram or a pharmaceutically acceptable salt thereof per gram of bead and a first polymer comprising about 20 mg to about 60 mg per gram of bead; a modified release coating comprising about 50 to about 300 mg per gram of bead; a second drug component comprising about 50 mg to about 150 mg escitalopram or a pharmaceutically acceptable salt thereof per gram of bead and a second polymer comprising about 5 mg to about 50 mg per gram of bead; and optionally, a top coating comprising about 5 mg to about 25 mg per gram of bead, wherein the oral dosage form has a dissolution rate of the active ingredient of about 70% to about 80% within about 4 hours to about 24 hours.

In exemplary embodiments, the first drug component comprises a core comprising about 675 mg sugar per gram of bead, about 105 mg escitalopram oxalate per gram of bead and the first polymer comprises about 37 mg hydroxypropyl cellulose per gram of bead; the modified release coating comprises about 163 mg surelease per gram of bead; the second drug component comprises about 105 mg escitalopram oxalate per gram of bead and the second polymer comprises about 21 mg hydroxypropyl cellulose per gram of bead; and the top coating comprises about 17 mg opadry clear per gram of bead.

### BRIEF DESCRIPTION OF THE DRAWINGS

FIG. 1 shows the pharmacokinetic profile for 10 mg escitalopram tablets, 8 mg escitalopram IR beads (calculated), modified release bead I, modified release bead II and modified release bead III.

FIG. 2 shows the dissolution profile for a slow release escitalopram bead (MR bead I) prepared in accordance with Example 3.

FIG. 3 shows the dissolution profile for the intermediate release escitalopram bead (MR bead II) prepared in accordance with Example 3.

FIG. 4 shows the dissolution profile for the fast release escitalopram bead (MR bead III) prepared in accordance with Example 3.

FIG. 5 shows the dissolution profile for the slow release escitalopram tablet (MR tablet I) prepared in accordance with Example 4.

FIG. 6 shows the dissolution profile for the intermediate release escitalopram tablet (MR tablet II) prepared in accordance with Example 4.

FIG. 7 shows the dissolution profile for the fast release escitalopram tablet (MR tablet III) prepared in accordance with Example 4.

FIG. 8 shows the dissolution profile of a unitary two-pulse tablet prepared in accordance with Example 5. The first pulse releases immediately in 0.1 N HCl solution. The second pulse releases less than 10% of the drug in 0.1 N HCl over the first 2 hours and releases after 2 hours in a phosphate buffer solution at a pH of 6.8.

FIG. 9 shows the calculated pharmacokinetic profile for an 8 mg dosage form of escitalopram comprised of 50% IR beads and 50% MR bead II and the pharmacokinetic profile for an IR dosage form of escitalopram.

FIG. 10 shows the calculated pharmacokinetic profile for an 8 mg dosage form of escitalopram comprised of 37.5% IR beads and 62.5% MR bead II and the pharmacokinetic profile for an IR dosage form of escitalopram.

FIG. 11 shows the calculated pharmacokinetic profile for an 8 mg dosage form of escitalopram comprised of 50% IR beads and 50% MR bead I and the pharmacokinetic profile for an IR dosage form of escitalopram.

FIG. 12 shows the calculated pharmacokinetic profile for an 8 mg dosage form of escitalopram comprised of 37.5% IR beads and 62.5% MR bead I and the pharmacokinetic profile for an IR dosage form of escitalopram.

### DETAILED DESCRIPTION OF THE INVENTION

The present invention relates to modified and pulsatile release pharmaceutical formulations of escitalopram and their use for the treatment of central nervous system (CNS) disorders, including mood disorders (e.g., major depressive disorder) and anxiety disorders (e.g., generalized anxiety disorder, social anxiety disorder, post traumatic stress disorder, obsessive compulsive disorder and panic disorder). In particular, the present invention provides modified release oral dosage forms of escitalopram or pharmaceutically acceptable salts thereof (preferably escitalopram oxalate) with enhanced pharmacokinetic properties. For example, it is desirable to provide an oral dosage form that provides the maximum amount of escitalopram to an individual, measured as the area under the plasma concentration-time curve (AUC₀₋ₜ and AUC_{0-∞}), while minimizing the maximum plasma concentration (Cₘₐₓ) that is produced. Moreover, it is desirable to provide a maximum plasma concentration (Cₘₐₓ) after a specific amount, i.e., control Tₘₐₓ. Thus, the present invention provides modified release oral dosage forms of escitalopram or pharmaceutically acceptable salts thereof with improved effectiveness in the treatment of central nervous system disorders and fewer side effects than prior escitalopram formulations.

According to some embodiments, the present invention provides oral dosage forms that include from about 2 mg to about 30 mg escitalopram or a pharmaceutically acceptable salt thereof, wherein the dosage form provides an in vivo plasma profile comprising a mean Tmax of more than about 6 hours, a mean Cmax of less than about 30 ng/ml and a mean AUC_{0-∞} of more than about 60 ng h/ml.

In further embodiments, the oral dosage forms include from about 5 mg to about 20 mg escitalopram or a pharmaceutically acceptable salt thereof. In still further embodiments, from about 4 mg to about 16 mg escitalopram or a pharmaceutically acceptable salt thereof are provided. In other embodiments, the dosage forms provide an in vivo plasma profile comprising a mean Tmax of more than about 8 hours. In other embodiments, the dosage forms provide an in vivo plasma profile comprising a mean Cmax of less than about 10.0 ng/ml. In still other embodiments, the dosage forms provide an in vivo plasma profile comprising a mean Cmax of less than about 5.0 ng/ml. In yet other embodiments, the dosage forms provide an in vivo plasma profile comprising a mean AUC_{0-∞} of more than about 120 ng h/ml. In still other embodiments, the dosage forms provide an in vivo plasma profile comprising a mean AUC_{0-∞} of more than about 150 ng h/ml. In other embodiments, the dosage forms provide an in vivo plasma profile comprising a mean AUC_{0-∞} of more than about 300 ng h/ml.

In exemplary embodiments, the dosage forms of the present invention comprise about 2 mg escitalopram or a pharmaceutically acceptable salt thereof and provides an in vivo plasma profile comprising a mean Tmax of more than about 6 hours, a mean Cmax of less than about 2 ng/ml and a mean AUC_{0-∞} of more than about 60 ng h/ml.

In other exemplary embodiments, the dosage forms of the present invention comprise about 4 mg escitalopram or a pharmaceutically acceptable salt thereof and provides an in vivo plasma profile comprising a mean Tmax of more than about 6 hours, a mean Cmax of less than about 4 ng/ml and a mean AUC_{0-∞} of more than about 120 ng h/ml.

In other exemplary embodiments, the dosage forms of the present invention comprise about 5 mg escitalopram or a pharmaceutically acceptable salt thereof and provides an in vivo plasma profile comprising a mean Tmax of more than about 6 hours, a mean Cmax of less than about 5 ng/ml and a mean AUC_{0-∞} of more than about 150 ng h/ml.

In other exemplary embodiments, the dosage forms of the present invention comprise about 10 mg escitalopram or a pharmaceutically acceptable salt thereof and provides an in vivo plasma profile comprising a mean Tmax of more than about 6 hours, a mean Cmax of less than about 10.0 ng/ml and a mean AUC_{0-∞} of more than about 300 ng h/ml.

In other exemplary embodiments, the dosage forms of the present invention comprise about 20 mg escitalopram or a pharmaceutically acceptable salt thereof and provides an in vivo plasma profile comprising a mean Tmax of more than about 6 hours a mean Cmax of less than about 20 ng/ml and a mean AUC_{0-∞} of more than about 600 ng h/ml.

In still other exemplary embodiments, the dosage forms of the present invention comprise about 30 mg escitalopram or a pharmaceutically acceptable salt thereof and provides an in vivo plasma profile comprising a mean Tmax of more than about 6 hours a mean Cmax of less than about 30 ng/ml and a mean AUC_{0-∞} of more than about 900 ng h/ml.

The dosage forms of the present invention may include modified or pulsatile release beads, tablets, and/or particles of escitalopram or a pharmaceutically acceptable salt thereof. Preferably, the beads, tablets, and/or particles are coated with a release modifying polymer. Suitable release modifying polymers include, but are not limited to, ethylcellulose, hydroxypropyl methylcellulose, acrylate based polymers, and mixtures thereof.

According to other embodiments, the present invention provides oral dosage forms that include about 2 to about 30 mg escitalopram or a pharmaceutically acceptable salt thereof wherein the dosage form has a dissolution rate of the active ingredient of about 70% to about 80% within about 4 hours to about 24 hours and provides an in vivo plasma profile comprising a mean Cₘₐₓ of less than about 30 ng/ml. In a further embodiment, the dosage form provides an in vivo plasma profile comprising a mean AUC_{0-∞} of more than about 120 ng h/ml. In another embodiment, the dosage form provides an in vivo plasma profile comprising a mean AUC_{0-∞} of more than about 150 ng h/ml.

For example, the dosage form may comprise about 2 mg escitalopram or a pharmaceutically acceptable salt thereof and an in vivo plasma profile comprising a mean AUC_{0-∞} of more than about 60 ng h/ml and a mean Cₘₐₓ of less than about 2 ng/ml. In other examples, the dosage form may comprise about 4 mg escitalopram or a pharmaceutically acceptable salt thereof and an in vivo plasma profile comprising a mean AUC_{0-∞} of more than about 120 ng h/ml and a mean Cₘₐₓ of less than about 4 ng/ml. In other examples, the dosage form may comprise about 5 mg escitalopram or a pharmaceutically acceptable salt thereof and an in vivo plasma profile comprising a mean AUC_{0-∞} of more than about 150 ng h/ml and a mean Cₘₐₓ of less than about 5 ng/ml. In another example, the dosage form may comprise about 10 mg escitalopram or a pharmaceutically acceptable salt thereof and an in vivo plasma profile comprising a mean AUC_{0-∞} of more than about 300 ng h/ml and a mean Cₘₐₓ of less than about 10 ng/ml. In yet another example, the dosage form may comprise about 20 mg escitalopram or a pharmaceutically acceptable salt thereof and an in vivo plasma profile comprising a mean AUC_{0-∞} of more than about 600 ng h/ml and a mean Cₘₐₓ of less than about 18 ng/ml. In other examples, the dosage form may comprise about 30 mg escitalopram or a pharmaceutically acceptable salt thereof and an in vivo plasma profile comprising a mean AUC_{0-∞} of more than about 900 ng h/ml and a mean Cₘₐₓ of less than about 30 ng/ml.

According to one embodiment, the oral dosage form is administered once a day and, upon ingestion by a patient, induces a statistically significant lower Cₘₐₓ for escitalopram or a pharmaceutically acceptable salt thereof in the plasma of the patient as compared to an immediate release dosage form containing the same amount of escitalopram or pharmaceutically acceptable salt thereof. Moreover, the dosage form may also provide bioavailability (AUC) of escitalopram that is substantially equivalent to that of an immediate release tablet containing the same form of escitalopram similarly administered once a day.

Preferably, the present dosage form is a once a day formulation, i.e., only administering once a day is required to provide the patient with a therapeutic effect over the entire day. One advantage of the present invention is the reduction of adverse events associated with prior escitalopram formulations. For example, using the dosage forms of the present invention, Cₘₐₓ-related adverse events may be reduced irrespective of the in vitro profile. Another possible advantage is that the dosage administered may be increased without increasing adverse events.

According to some embodiments, the present invention provides composite dosage forms that include an immediate release component and a modified release component, wherein the immediate release component comprises a first active ingredient comprising about 2 to about 30 mg escitalopram or a pharmaceutically acceptable salt thereof, wherein about 80% of the first active ingredient dissolves within about the first 4 hours following entry of the dosage form into a use environment and wherein the modified release component comprises a second active ingredient comprising about 2 to about 30 mg escitalopram or a pharmaceutically acceptable salt thereof, wherein about 70% to about 80% of the second active ingredient dissolves within about 4 hours to about 24 hours following entry of the dosage form into the use environment.

The composite dosage forms may includes beads and/or tablets of escitalopram having at least two different release profiles (i.e., at least two separate pulses of escitalopram or a pharmaceutically acceptable salt thereof). The number of pulses released by the dosage form preferably ranges from one to four, more preferably from one to three, and even more preferably is two. According to one embodiment, the pulsatile dosage form of the present invention comprises an immediate release pulse followed by one or more delayed release pulses occurring later in time.

Pulsatile release means that the escitalopram is released in one or more pulses, each pulse having a unique dissolution profile. Each pulse may be released at a different time or under different environmental conditions after the dosage form is administered. Thus, predetermined amounts of escitalopram may be individually released after administration. A pulsatile dosage form with a multiphase release containing at least one modified release formulation may be employed to attain a combination of release rates that are suitable for specific therapeutic objectives. For example, a portion of the drug can be released immediately, followed by an extended release of escitalopram. The dosage form may include two or three groups of drug-containing particles or beads, i.e., each group of particles or beads has a different drug release profile. The number of pulses and the amount of drugs released preferably will result in a Tₘₐₓ of about 5 to about 35 hours. The individual dosage units (such as beads and particles) can be compacted in a single tablet or placed in a capsule. For example, different layers of the tablet may represent different dosage units. Drug-containing particles or drug-containing beads can also be compressed together into a single tablet using conventional tabletting means. One skilled in art will recognize that other dosage forms may also be developed.

Pulsatile release profiles can be achieved with dosage forms such as capsules or tablets, which contain two or more drug-containing dosage units. Preferably, at least two of the dosage units provide different drug release profiles. Each dosage unit can be a tablet (e.g., compressed or molded), bead, or particle. Suitable pulsatile systems are described in U.S. Pat. Nos. 6,217,904, 6,555,136, 6,793,936, 6,627,223, 6,372,254, 6,730,321, 6,500,457, 4,723,958, 5,840,329, 5,508,040, and 5,472,708 and U.S. Patent Application Publication Nos. US 2003-124196, US 2004-028729, and US 2003-0133978.

For example, a capsule may contain two or three tablets. The first tablet in the capsule may release escitalopram substantially quickly following ingestion of the dosage form, while the second tablet in the capsule releases escitalopram more slowly following ingestion, and an optional third tablet could provide escitalopram release even more slowly. While the dosage form will not generally include more than three tablets, dosage forms housing four or more tablets are within the scope of the present invention. One skilled in the art will appreciate that the capsule release profile can be achieved by the combination of release rates and strengths of each tablet.

Tablets in accordance with this invention can be prepared by conventional mixing, comminution, and tabletting techniques that are well known in the pharmaceutical formulations industry. The modified-release tablet, for example, may be fabricated by direct compression by punches and dies fitted to a rotary tabletting press, ejection or compression molding, granulation followed by compression, or forming a paste and extruding the paste into a mold or cutting the extrudate into short lengths.

When polyethylene oxide in combination with hydroxypropyl methylcellulose or ethyl cellulose are used, the dissolution rates can be much slower than the modified release rate targeted. The slow release is because hydrophobic matrix tablets that are formed release the drug by mechanism of polymer erosion. Since the erosion from a hydrophobic matrix is slow, the dissolution rate of the readily soluble active ingredient is also slow. Lactose, or microcrystalline cellulose may be used as a filler ingredient to modulate the release rates of the tablet.

When tablets are made by direct compression, the addition of lubricants may be helpful and is sometimes important to promote powder flow and to prevent capping of the tablet (the breaking off of a portion of the tablet) when the pressure is relieved. Useful lubricants include magnesium stearate, and hydrogenated vegetable oil (preferably hydrogenated and refined triglycerides of stearic and palmitic acids). In a preferred embodiment, the lubricant is magnesium stearate. For the 24-hour release formulations, the magnesium stearate preferably is present in amounts ranging from about 0.25% w/w to about 5% w/w, preferably from about 0.5% w/w to about 4% w/w. Additional excipients may be added to enhance tablet hardness, powder flowability, and tablet friability and to reduce adherence to the die wall.

In other embodiments, the dosage form may comprise beads of escitalopram or a pharmaceutically acceptable salt thereof. Beads offer advantages over conventional solid oral modified release dosage forms, such as tablets. Beads are dose proportional, i.e., different doses may be obtained by using different proportions and amounts of beads. Beads also enable a variety of dissolution profiles by mixing one or more types of beads having different dissolution properties or by using multi-layer coatings. Such multiple dissolution profiles may not be possible using modified release tablet formulations. Beads also enable a wide range of drug loading. One skilled in art will recognize that different dissolution profiles can be obtained by combining different compositions of beads or tablets.

In another embodiment, the dosage form may be a matrix tablet or matrix bead containing escitalopram or a pharmaceutically acceptable salt thereof. In a matrix-controlled release approach, lipophilic substances, e.g., higher alcohols, waxes, or insoluble thermoplastic materials, are employed. The release is controlled by the rate of diffusion of the active ingredient into the surrounding medium and, if the matrix itself is erodible, by the rate of its erosion.

In compositions comprising a hydrophilic matrix, the matrix may be composed of an insoluble hydrophilic polymer, such as a cellulose ester, carboxyvinyl ester, or acrylic or methacrylic ester. On contact with biological fluids, the matrix becomes hydrated and swells, forming a very dense network of polymers, through which polymers the soluble active principles diffuse. Furthermore, lipids, in particular polyethylene oxide can be added to modulate the matrix swelling. These compositions can be obtained by granulation and then compression of the mixture formed of the polymer, active principles and various adjuvants.

The modified release dosage units for the pulsatile and sustained release formulations can be prepared, for example, by coating a drug or a drug-containing composition with coating material, such as a polymeric material. When a coating is used to provide delayed release dosage units, particularly preferred coating materials include, but are not limited to, bioerodible, gradually hydrolyzable and/or gradually water-soluble polymers. The “coating weight,” or relative amount of coating material per dosage unit, generally dictates the time interval between ingestion and drug release.

Suitable coating materials for effecting release include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose hexahydrophthalate, cellulose acetate hexahydrophthalate, and carboxymethylcellulose sodium; acrylic acid polymers and copolymers preferably formed from acrylic acid, methacrylic acid, acrylic acid alkyl esters, methacrylic acid alkyl esters, and the like, e.g. copolymers of acrylic acid, methacrylic acid, methyl acrylate (sold as Eudragit and Acryl-EZE®), ethyl acrylate, methyl methacrylate and/or ethyl methacrylate, with a terpolymer of ethyl acrylate, methyl methacrylate and trimethylammonioethyl methacrylate chloride (sold under the tradename Eudragit) particularly preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac, ammoniated shellac, shellac-acetyl alcohol, and shellac n-butyl stearate.

To bring about the desired pulsatile and modified release profile for a dosage form comprised of encapsulated tablets, the first tablet is provided with a specified amount of material, the second tablet is provided with more or different material(s) to delay or extend release of the active ingredient beyond that of the first tablet, and subsequent tablets have more or different material(s) to further delay release of the active ingredient beyond each previous tablet. One skilled in art will recognize that the modified release tablets and beads, e.g., the second pulse, can be further coated with active drug which generates the first pulse. The tablet may also be coated with release modifying polymers. Analogously, for encapsulated dosage forms, in which the drug-containing dosage units are beads or particles, a first fraction of beads or particles is provided with a specified amount material, a second fraction is provided with more coating material(s), or different material, to delay release of the active ingredient beyond that of the first fraction, and subsequent fractions are coated with material(s) to further delay release of the active ingredient beyond each previous fraction.

For example, when the dosage form contains two tablets (or, analogously, two groups of drug-containing particles or beads), the first tablet, which releases drug substantially quickly following ingestion of the dosage form, such that less than about 50% of drug is released, based on 100% total weight of the first tablet. The second tablet, which releases escitalopram more slowly following ingestion, may then release the remainder of the drug. The preferred polymer materials may be determined by those skilled in the art by evaluating individual release profiles for dosage units prepared with different quantities of various coating materials. One skilled in the art will appreciate that the weight gain depends on the coating materials and polymers used.

Alternatively, the delayed release dosage units, e.g., tablets, beads, or particles, may be formulated by coating the drug within a suitable material such as a plastic, a hydrophilic polymer, or a fatty compound. The insoluble plastic matrices may be comprised of, for example, polyvinyl chloride or polyethylene. Hydrophilic polymers useful for providing a matrix for a delayed release dosage unit include, but are not limited to, those described above as suitable coating materials. Once the drug is mixed with the matrix material, the mixture may be compressed into tablets or processed into individual drug-containing particles.

The individual dosage units may be provided with colored coatings, with a single color used to identify a tablet, bead, or particle fraction having a corresponding delayed release profile. For example, a blue coating may be used for the immediate release tablet, bead, or particle fraction, a red coating may be used for the “medium” release tablet, bead, or particle fraction, and the like. In this way, errors during manufacture can be easily avoided. The color is introduced by incorporating a pharmaceutically acceptable colorant into the coating during coating preparation or tabletting. The colorant may be either natural or synthetic. Natural colorants include pigments such as chlorophyll, anattenes, beta-carotene, alizarin, indigo, rutin, hesperidin, quercitin, carminic acid, and 6,6′-dibromoindigo. Synthetic colorants are dyes, including both acidic dyes and basic dyes, such as nitroso dyes, nitro dyes, azo dyes, oxazines, thiazines, pyrazolones, xanthenes, indigoids, anthraquinones, acridines, rosanilines, phthaleins, quinolines. For example, a dye or pigment could be incorporated during preparation of the coating solution.

For encapsulated tablets, the weight of each individual tablet in the capsule is typically in the range of about 50 mg to about 700 mg, preferably in the range of about 60 mg to about 600 mg. One skilled in the art will appreciate that weight of tablets can differ from those above based on filler and process selection. The individual tablets can be prepared by conventional means. A preferred method for forming tablets herein is by direct compression of a powdered, crystalline, or granular drug-containing composition, alone or in combination with diluents, binders, lubricants, disintegrants, colorants, or the like. Compressed tablets can also be prepared by wet-granulation or dry-granulation processes. Tablets may also be molded rather than compressed, starting with a moist material containing a suitable water-soluble lubricant. Drug-containing particles or beads may also be prepared by conventional means, such as with a fluid dispersion.

Conventional coating procedures and equipment may be used to coat the dosage units, e.g., the drug-containing tablets, beads, or particles. For example, a delayed release coating composition may be applied using a coating pan, an airless spray technique, or fluidized bed coating equipment. Materials, equipment and processes for preparing tablets, beads, drug particles, and delayed release dosage forms are described in Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6^{th} Ed. (Media, Pa.: Williams & Wilkins, 1995).

Optional components present in the individual drug-containing dosage units include, but are not limited to, diluents, binders, lubricants, disintegrants, stabilizers, antioxidants surfactants, and coloring agents.

Diluents (also referred to as “fillers”) are typically included to increase the bulk of a tablet so that a practical size is provided for compression. Suitable diluents include, but are not limited to, dicalcium phosphate dihydrate, calcium sulfate, lactose, cellulose, kaolin, mannitol, sodium chloride, dry starch, hydrolyzed starches, silicon dioxide, titanium oxide, alumina, talc, microcrystalline cellulose, powdered sugar, and mixtures thereof.

Binders are used to impart cohesive qualities to a tablet formulation, and thus ensure that a tablet remains intact after compression. Suitable binder materials include, but are not limited to, starch (including corn starch and pre-gelatinized starch), gelatin, sugars (including sucrose, glucose, dextrose, lactose and sorbitol), polyethylene glycol, waxes, natural and synthetic gums, e.g., acacia, tragacanth, sodium alginate, polyvinylpyrrolidone, celluloses, and Veegum, and synthetic polymers such as polymethacrylates and polyvinylpyrrolidone (PVP), and mixtures thereof.

Lubricants are used to facilitate tablet manufacture. Examples of suitable lubricants include, but are not limited to, magnesium stearate, calcium stearate, stearic acid, glyceryl behenate, and polyethylene glycol. Preferably, a dosage unit contains no more than approximately 1 wt. % (relative to dosage unit weight) of lubricant.

Disintegrants are used to facilitate tablet disintegration or “breakup” after administration. Suitable disintegrants include, but are not limited to, starches, clays, celluloses, algins, gums, crosslinked polymers, and mixtures thereof.

Surfactants may be anionic, cationic, amphoteric or nonionic surface active agents. Suitable anionic surfactants include, but are not limited to, those containing carboxylate, sulfonate and sulfate ions, associated with cations such as sodium, potassium and ammonium ions. Other suitable surfactants include, but are not limited to, long alkyl chain sulfonates and alkyl aryl sulfonates such as sodium dodecylbenzene sulfonate; dialkyl sodium sulfosuccinates, such as sodium bis-(2-ethylhexyl)-sulfosuccinate; and alkyl sulfates, such as sodium lauryl sulfate.

If desired, the tablets may also contain minor amounts of nontoxic auxiliary substances such as wetting or emulsifying agents, pH buffering agents, preservatives, and the like.

As noted earlier herein, the individual drug tablets, beads or particles are, in one embodiment, contained within a closed capsule. The capsule material may be either hard or soft, and as will be appreciated by those skilled in the art of pharmaceutical science, typically comprises a tasteless, easily administered and water soluble compound, such as gelatin, starch, or cellulose. A preferred capsule material is gelatin. The capsules are preferably sealed, such as with gelatin bands. See, e.g., Remington: The Science and Practice of Pharmacy, 20^{th} Edition (Easton, Pa.: Mack Publishing Co., 2000), which describes materials and methods for preparing encapsulated pharmaceuticals designed to dissolve shortly after ingestion.

According to some embodiments, the present invention provides oral dosage forms comprising a plurality of beads, each bead comprising a core having a diameter from about 1 μm to about 1000 μm; an active ingredient comprising about 2 to about 30 mg escitalopram or a pharmaceutically acceptable salt thereof; and a modified release coating, wherein the oral dosage form has a dissolution rate of the active ingredient of about 70% to about 80% within about 4 hours to about 24 hours; and wherein the dosage form provides an in vivo plasma profile comprising an in vivo plasma profile comprising a mean Cₘₐₓ of less than about 30 ng/ml. In a further embodiment, the dosage forms provide an in vivo plasma profile comprising a mean AUC_{0-∞} of more than about 60 ng h/ml.

In exemplary embodiments, the release modifying polymer may include, but is not limited to, ethylcellulose (Surelease®), methacrylate (Eudragit®), methacrylic acid copolymer type C (Acryl-eze®), and mixtures thereof. In some embodiments, the oral dosage form may include a top coating coated on the release modifying polymer layer. For example, the top coating may include, but is not limited to, HPMC (Opadry®), HPC (Klurel®), Eudragit® RL, Eudragit® E100, Eudragit® E 12.5, Eudragit® E PO, Eudragit® NE, and mixtures thereof.

According to other embodiments, the present invention provides oral dosage forms comprising a plurality of beads, wherein each bead comprises: a first drug component comprising a core comprising about 500 to about 800 mg sugar per gram of bead, about 30 mg to about 300 mg escitalopram or a pharmaceutically acceptable salt thereof per gram of bead and a first polymer comprising about 20 mg to about 60 mg per gram of bead; a modified release coating comprising about 50 to about 300 mg per gram of bead; a second drug component comprising about 50 mg to about 150 mg escitalopram or a pharmaceutically acceptable salt thereof per gram of bead and a second polymer comprising about 5 mg to about 50 mg per gram of bead; and optionally, a top coating comprising about 5 mg to about 25 mg per gram of bead, wherein the oral dosage form has a dissolution rate of the active ingredient of about 70% to about 80% within about 4 hours to about 24 hours.

In an exemplary embodiment, the first drug component comprises a core comprising about 675 mg sugar per gram of bead, about 105 mg escitalopram oxalate per gram of bead and the first polymer comprises about 37 mg hydroxypropyl cellulose per gram of bead; the modified release coating comprises about 163 mg surelease per gram of bead; the second drug component comprises about 105 mg escitalopram oxalate per gram of bead and the second polymer comprises about 21 mg hydroxypropyl cellulose per gram of bead; and the top coating comprises about 17 mg opadry clear per gram of bead.

According to some embodiments, the present invention provides capsules that include one or more of the beads provided herein. For example, the present invention provides drug capsules that includes about 2 mg escitalopram or a pharmaceutically acceptable salt thereof and provide an in vivo plasma profile comprising a mean Tmax of more than about 6 hours; a mean Cmax of less than about 2 ng/ml; and a mean AUC_{0-∞} of more than about 60 ng h/ml.

In other examples, the present invention provides drug capsules that include about 4 mg escitalopram or a pharmaceutically acceptable salt thereof and provide an in vivo plasma profile comprising a mean Tmax of more than about 6 hours; a mean Cmax of less than about 4 ng/ml; and a mean AUC_{0-∞} of more than about 120 ng h/ml.

In other examples, the present invention provides drug capsules that include about 5 mg escitalopram or a pharmaceutically acceptable salt thereof and provide an in vivo plasma profile comprising a mean Tmax of more than about 6 hours; a mean Cmax of less than about 5 ng/ml; and a mean AUC_{0-∞} of more than about 150 ng h/ml. In yet other examples, the present invention provides drug capsules that include about 10 mg escitalopram or a pharmaceutically acceptable salt thereof and provide an in vivo plasma profile comprising a mean Tmax of more than about 6 hours; a mean Cmax of less than about 10.0 ng/ml; and a mean AUC_{0-∞} of more than about 300 ng h/ml. In other examples, the present invention provides drug capsules that include about 20 mg escitalopram or a pharmaceutically acceptable salt thereof and provide an in vivo plasma profile comprising a mean Cmax of less than about 20 ng/ml; and a mean AUC_{0-∞} of more than about 600 ng h/ml. In still other examples, the present invention provides drug capsules that include about 30 mg escitalopram or a pharmaceutically acceptable salt thereof and provide an in vivo plasma profile comprising a mean Cmax of less than about 30 ng/ml; and a mean AUC_{0-∞} of more than about 900 ng h/ml.

According to other embodiments, the present invention provides methods of treating patients suffering from an adverse event brought on by treatment with an antidepressant administered in a dosage form other than a dosage form containing pulsatile release escitalopram or a pharmaceutically acceptable salt thereof (preferably, escitalopram oxalate). Examples of such adverse events include, but are not limited to, nausea, insomnia, somnolence, increased sweating, fatigue, or a combination thereof. The method includes (a) discontinuing treatment with the antidepressant; and (b) treating the patient with a pulsatile dosage form of the present invention. According to one embodiment, the antidepressant is an immediate release dosage form, preferably an immediate release escitalopram oxalate dosage form.

According to other embodiments, the present invention provides methods for treating patients suffering from sexual dysfunction brought on by treatment with an antidepressant administered in a dosage form other than a dosage form containing pulsatile release escitalopram or a pharmaceutically acceptable salt thereof (preferably, escitalopram oxalate). Examples of such sexual dysfunction include, but are not limited to, ejaculation disorder, anorgasmia, and/or decreased libido. The method includes (a) discontinuing treatment with the antidepressant; and (b) treating the patient with a pulsatile dosage form of the present invention. According to one embodiment, the antidepressant is a modified release escitalopram oxalate dosage form.

According to other embodiments, the present invention provides methods of treating a CNS disorder in a patient in need thereof by administering an effective amount of the dosage form of the present invention to the patient. CNS disorders that can be treated with the dosage form of the present invention include, but are not limited to, major depressive disorder, generalized anxiety disorder, social anxiety disorder, post traumatic stress disorder, obsessive compulsive disorder, panic disorder (including panic attacks), acute stress disorder, eating disorders (such as binge eating, bulimia, anorexia, and obesity), phobias, dysthymia, premenstrual syndrome, premenstrual dysphoric disorder, cognitive disorders, impulse control disorders, mood disorders, neurotic disorders, acute stress disorders, attention deficit hyperactivity disorder, and drug abuse. The dosage form can also effectively treat patients who have failed to respond to initial treatment with a conventional SSRI, especially in patients with major depressive disorder who have failed to respond to initial treatment with a conventional SSRI. The dosage form can also be used to treat or reduce suicidal thoughts in a patient in need thereof, and improve disability-free survival following stroke.

### DEFINITIONS

The term “escitalopram” as used herein includes 1-[3-(dimethyl-amino)propyl]-1-(p-fluorophenyl)-5-phthalancarbonitrile preferably containing less than 3%, 2%, 1%, 0.5%, or 0.2% by weight of its R-enantiomer (based on 100% total weight of 1-[3-(dimethyl-amino)propyl]-1-(p-fluorophenyl)-5-phthalancarbonitrile)—i.e., S-citalopram having an enantiomeric purity of more than 97, 98, 99, 99.5, or 99.8% by weight. For example, the escitalopram can contain between 3% and 0.2% by weight of its R-enantiomer (based on 100% total weight of 1-[3-(dimethyl-amino)propyl]-1-(p-fluorophenyl)-5-phthalancarbonitrile).

Pharmaceutically acceptable salts of escitalopram include, but are not limited to, acid addition salts formed with organic and inorganic acids. Examples of such organic acids are maleic, fumaric, benzoic, ascorbic, pamoic, succinic, oxalic, salicylic, methanesulfonic, ethanedisulfonic, acetic, propionic, tartaric, citric, gluconic, lactic, malic, mandelic, cinnamic, citraconic, aspartic, stearic, palmitic, itaconic, glycolic, p-amino-benzoic, glutamic, benzene sulfonic and theophylline acetic acid, as well as the 8-halotheophyllines, for example 8-bromotheophylline. Examples of such inorganic acids are hydrochloric, hydrobromic, sulfuric, sulfamic, phosphoric, and nitric acids. Preferred pharmaceutically acceptable salts of escitalopram include, but are not limited to, escitalopram oxalate and escitalopram hydrobromide. The term “escitalopram” also includes polymorphs, hydrates, solvates, and amorphous forms of escitalopram and its pharmaceutically acceptable salts. Escitalopram and pharmaceutically acceptable salts thereof can be prepared as described in U.S. Pat. Nos. Re. 34,712 and 6,566,540 and International Publication Nos. WO 03/000672, WO 03/006449, WO 03/051861, and WO 2004/083197. Crystals of escitalopram oxalate and escitalopram hydrobromide such as those described in International Publication No. WO 03/011278, U.S. Patent Application Publication No. 2004/0167209, and U.S. patent application Ser. Nos. 10/851,763 and 10/948,594 can also be used. The comparative escitalopram “immediate release” tablets referred to herein are preferably those of U.S. Food and Drug Administration Approved New Drug Application No. 21-323 of equal amount (5 mg, 10 mg, or 20 mg escitalopram). Unless otherwise indicated, amounts of escitalopram refer to amounts of escitalopram free base. One skilled in the art will understand that for any desired amount of escitalopram a specific amount of a particular salt must be used. For example, 1.28 mg of escitalopram oxalate is equivalent to 1.0 mg of escitalopram free base. Determination of the amount of a particular salt that must be used to provide a desired amount of escitalopram free base is well within the skill of one of the art.

A “therapeutically effective amount” means the amount of an active ingredient that, when administered to a mammal for treating a state, disorder, or condition is sufficient to effect such treatment. The “therapeutically effective amount” will vary depending on the active ingredient, the state, disorder, or condition to be treated and its severity, and the age, weight, physical condition, and responsiveness of the mammal to be treated. According to one embodiment of the present invention, a therapeutically effective amount of escitalopram is an amount effective to treat CNS disorders, including mood disorders, major depressive disorder, generalized anxiety disorder, social anxiety disorder, post traumatic stress disorder, and panic disorder, including panic attacks.

The term “pharmaceutically acceptable” means biologically or pharmacologically compatible for in vivo use in animals or humans, and preferably means approved by a regulatory agency of the Federal government or of a state government or listed in the U.S. Pharmacopeia or other generally recognized pharmacopeia for use in animals, and more particularly in humans.

As used herein, the terms “treat,” “treatment,” and “treating” refer to one or more of the following:

(a) relieving or alleviating at least one symptom of a disorder in a subject, including for example, CNS disorders, including mood disorders, major depressive disorder, generalized anxiety disorder, social anxiety disorder, post traumatic stress disorder, and panic disorder, including panic attacks;

(b) relieving or alleviating the intensity and/or duration of a manifestation of a disorder experienced by a subject including, but not limited to, those that are in response to a given stimulus (e.g., pressure, tissue injury, cold temperature, etc.);

(c) arresting, delaying the onset (i.e., the period prior to clinical manifestation of a disorder) and/or reducing the risk of developing or worsening a disorder.

The term “panic attacks” includes, but is not limited to, any disease that is associated with panic attacks including panic disorder, specific phobias, social phobia, and agoraphobia, in which panic attacks occur. These disorders are further defined in the DSM IV. AMERICAN PSYCHIATRIC ASSOCIATION, DIAGNOSTIC AND STATISTICAL MANUAL OF MENTAL DISORDERS (4th ed. 1994). A panic attack is a discrete period in which there is a sudden onset of intense apprehension, fearfulness or terror, often associated with feelings of impending doom. During the attack, symptoms such as palpitations, sweating, trembling, sensations of shortness of breath, feelings of choking, chest pain or discomfort, nausea, feelings of dizziness, feelings of unreality, fear of losing control or going crazy, fear of dying, paresthesias and chills or hot flashes are present.

Panic disorders are characterized by recurrent unexpected panic attacks about which there is a persistent concern. Agoraphobia is anxiety about, or avoidance of, places or situations from which escape might be difficult or in which help may not be available in the event of a panic attack. Specific phobia and social phobia (together formerly “simple phobia”) are characterized by marked and persistent fear that is excessive or unreasonable, cued by the presence or anticipation of a specific object or situation (flying, heights, animals, seeing blood, etc.) or social performance situations.

The disorders in which panic attacks occur are differentiated from each other by the predictability of the occurrence of the attacks. For example, in panic disorder the attacks are unpredictable and not associated with any particular event, whereas in specific phobia the attacks are triggered by specific stimuli.

The phrase “treatment of panic disorder” can include a reduction in the number or prevention of panic attacks and/or relief of the severity of the panic attacks.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean a range of up to 10%, preferably up to 5%.

The term “immediate release” is defined herein as release of more than 80% by weight of the escitalopram in about 30 minutes. USP 24/NF 19 1088 2000:2051. The dissolution testing time is generally 30 to 60 minutes. Typical specifications for the amount of active ingredient dissolved, expressed as a percentage of the labeled content (Q), are in the range of 80% dissolved; FDA guidance for Industry: Dissolution Testing of IR Solid dosage forms, August 1997, p. 5, suggests the limit of NLT 85% in 60 minutes or less.

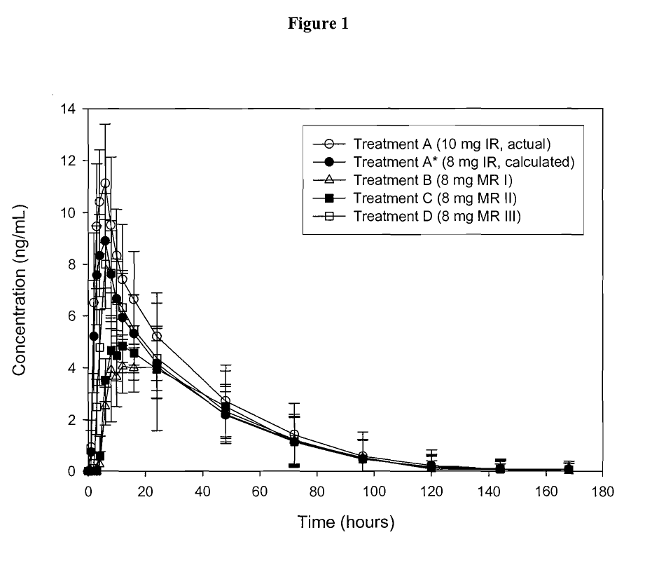

The terms “delayed release,” “sustained release,” and “modified release,” refer to the release of an active ingredient over an extended period of time leading to lower peak plasma concentrations and a prolonged Tₘₐₓ as compared to “immediate release” formulations. These terms also include release over a period of time via a series of immediate release pulses. The pharmacokinetic profile for 20 mg Lexapro® tablets (immediate release escitalopram oxalate tablets) is shown in FIG. 1. The peak plasma concentration was observed approximately 2-4 hours following administration.

The term “bioavailability” refers to the rate and extent to which the active ingredient or active moiety, e.g., escitalopram, is absorbed from a drug product and becomes systematically available.

The term “entry into a use environment” means contact of a formulation of the invention with the gastric fluids of the patient to whom it is administered, or with a fluid intended to simulate gastric fluid.

The term “pulsatile” as used herein refers to a plurality of escitalopram doses that are released at spaced apart time intervals.

The pharmacokinetic parameters described herein include area under the plasma concentration-time curve (AUC₀₋ₜ and AUC_{0-∞}), maximum plasma concentration (Cₘₐₓ), time of maximum plasma concentration (Tₘₐₓ) and terminal elimination half-life (T_{1/2}). The time of maximum concentration, Tₘₐₓ, was determined as the time corresponding to Cₘₐₓ. Area under the plasma concentration-time curve up to the time corresponding to the last measurable concentration (AUC₀₋ₜ) was calculated by numerical integration using the linear trapezoidal rule as follows:

$$AUC \text{?} = \text{?} 0.5 \cdot ( C \text{?} + C \text{?} ) - ( t \text{?} - t \text{?} ) \\ \text{?} \text{indicates text missing or illegible when filed} \qquad Eq . 1$$

where Cᵢ is the plasma memantine concentrations at the corresponding sampling time point tᵢ and n is the number of time points up to and including the last quantifiable concentration. Estimates of the terminal half-life (T_{1/2}) were calculated using the following equation:

$${T}_{1 / 2} = \frac{0.693}{{λ}_{2}} \qquad Eq . 2$$

where λ_{Z} is the terminal elimination rate constant.

The area under the plasma concentration-time curve from time zero to infinity was calculated according to the following equation:

$${AUC}_{0 - ∞} = {AUC}_{0 - t} + \frac{{C}_{last}}{{λ}_{z}} \qquad Eq . 3$$

where Cₗₐₛₜ is the last measurable concentration.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure. The invention is therefore to be limited only by the terms of the claims, along with the full scope of equivalents to which the claims are entitled. All parts and percentages are given by weight unless otherwise indicated.

### Example 1

### Immediate Release Tablets

Escitalopram oxalate is currently sold and marketed in the United States as Lexapro® for the treatment of major depressive disorder and generalized anxiety disorder. Lexapro® is available in 5, 10, and 20 mg immediate release (IR) tablets (as an oxalate salt). Examples of escitalopram oxalate IR tablets formulations are provided in table 1. The strength of the listed IR tablets range from 2.5 to 40 mg of escitalopram per tablet (calculated based on the weight of escitalopram free base). Table 1B shows the pharmacokinetic parameters (Cmax, AUC and Tmax) for immediate release escitalopram tablets (10 mg tablets were used and the data extrapolated to determine 2, 4, 5, 8, 15, 16, 20, 25 and 30 mg dosages). FIG. 1 shows the pharmacokinetic profile for 10 mg escitalopram tablets, 8 mg escitalopram IR beads (calculated), modified release bead I, modified release bead II and modified release bead III.

TABLE 1
Immediate Release formulations of Escitalopram

| | 2.5 mg | 4 mg | 5 mg | 10 mg | 20 mg | 40 mg |
|---|---|---|---|---|---|---|
| | Tablet | Tablet | Tablet | Tablet | Tablet | Tablet |
| Ingredient | (mg/Tablet) | (mg/Tablet) | (mg/Tablet) | (mg/Tablet) | (mg/Tablet) | (mg/Tablet) |
| S-citalopram | 3.2 | 5.1 | 6.4 | 12.8 | 25.6 | 51.2 |
| oxalate* | | | | | | |
| Talc, USP | 7.0 | 11.2 | 14.0 | 14.0 | 14.0 | 14.0 |
| Silicified | 109.8 | 175.7 | 219.6 | 213.2 | 200.4 | 174.8 |
| Macrocrystalline | | | | | | |
| Cellulose, NF | | | | | | |
| Croscarmellose | 4.5 | 7.2 | 9.0 | 9.0 | 9.0 | 9.0 |
| Sodium, NF | | | | | | |
| Magnesium | 0.5 | 0.8 | 1.0 | 1.0 | 1.0 | 1.0 |
| Stearate, NF | | | | | | |
| Total Core (mg) | 125 | 200 | 250 | 250 | 250 | 250 |

*1.28 mg of oxalate salt is equivalent to 1.0 mg of escitalopram base

TABLE 1B
Pharmokinetic parameters for immediate release escitalopram tablets

| | Dosage (mg) | | | | | | | | | |
|---|---|---|---|---|---|---|---|---|---|---|
| | 2 | 4 | 5 | 8 | 10 | 15 | 16 | 20 | 25 | 30 |
| Cmax | 2.3 | 4.6 | 5.8 | 9.2 | 11.5 | 17.3 | 18.4 | 23.0 | 28.8 | 34.5 |
| AUC 24 | 67.8 | 135.5 | 169.4 | 271.0 | 338.8 | 508.1 | 542.0 | 677.5 | 846.9 | 1016.3 |
| AUC | 80.3 | 160.5 | 200.6 | 321.0 | 401.3 | 601.9 | 642.0 | 802.5 | 1003.1 | 1203.8 |
| Tmax | 5 | 5 | 5 | 5 | 5 | 5 | 5 | 5 | 5 | 5 |

The immediate release (IR) tablets can be prepared as follows. Charge a PK twin shell blender with the screened drug substance and talc, USP and mix. Add screened silicified microcrystalline cellulose, NF and croscarmellose sodium, NF into the twin shell blender and mix. Add screened magnesium stearate, NF into the twin shell blender and mix. The blend is then compressed on a rotary tablet press to the specified core tablet weight. For IR tablets, the core tablets are film-coated in a perforated coating pan with OPADRY® White dispersion to an approximate weight gain of 2%.

An example of an 8 mg escitalopram oxalate IR tablet formulation is provided in Table 2.

TABLE 2
Escitalopram Nontrade Tablets, 8 mg

| Material | mg/tablet |
|---|---|
| S-citalopram Oxalate* | 10.2 |
| Talc, USP | 14.0 |
| Silicified Microcrystalline | 215.8 |
| Cellulose, NF | |
| Croscarmellose Sodium, NF | 9.0 |
| Magnesium Stearate, NF | 1.0 |
| Total cores | 250.0 |

*1.28 mg of oxalate salt is equivalent to 1.0 mg of escitalopram base

### Example 2

### Immediate Release Beads

Immediate release beads may be prepared using formulations of escitalopram oxalate by layering sugar spheres with the active drug (Table 3).

TABLE 3
Immediate release escitalopram beads

| | mg/g | |
|---|---|---|
| Ingredient | (range) | mg/g |
| S-citalopram Oxalate* | 30-300 | 128 |
| Binder: | 3-75 | 46 |
| Hydroxyproply Cellulose (HPC), Povidone or | | |
| Hydroxypropyl methyl cellulose (HPMC) | | |
| Talc | 0-10 | 0 |
| Sugar Spheres, or Micro Crystalline | 750-900 | 826 |
| Cellulose beads | | |
| Purified Water** | — | — |
| Total | 1000 | 1000 |

**1.28 mg of oxalate salt is equivalent to 1.0 mg of escitalopram base
**Purified water is removed during the process

One skilled in art will recognize that additional excipients, such as, antioxidants, pH modifiers may also be added.

The process for manufacturing the immediate release beads includes mixing the HPC binder (or PVP) with water and stirring until dissolved. The escitalopram oxalate is added and mixing continues for 15 minutes. Optionally, Talc is added and mixing is continued for at least 30 minutes to form a suspension. Pre-warmed sugar spheres USP are coated with a layer of the suspension using a fluidized bed coater (such as GPCG3, manufactured by Glatt Fluid Air, Ramsey, N.J.) using the following process parameters (for batch size=1.0-3.0 Kg): Product temperature=35-55° C.; Air flow=200-350 m³/h; Spray rate=9-42 gm/min; Atomization pressure=1.5-2.0 bar. The coated beads are dried for about 15 minutes in the fluidized bed and then discharged into appropriate storage containers. One skilled in art will recognize that other suitable process parameters are also acceptable. Optionally, the drug layered beads can be further coated with hydroxypropyl cellulose (HPC). One skilled in art will recognize that other methods of preparing the beads, such as extrusion spheronization, can also be used with appropriate excipients.

Immediate release bead formulations with 100 mg/g escitalopram and 200 mg/g escitalopram are shown in Tables 4 and 5, respectively.

TABLE 4
Escitalopram IR Beads, 100 mg/g

| Process stage | | Material | mg/g |
|---|---|---|---|
| 1. | Drug | S-citalopram Oxalate micronized* | 128.0 |
| | layering | Sugar Spheres, USP | 826.4 |
| | | Hydroxypropyl Cellulose, NF | 25.6 |
| | | Purified Water, USP | 0 |
| 2. | Top | Hydroxypropyl Cellulose | 20.0 |
| | coating | Purified Water, USP | 0 |
| | | Total MR beads | 1000.0 |

*1.28 mg of oxalate salt equiv. to 1 mg of base.

TABLE 5
Escitalopram IR Beads, 200 mg/g

| Process stage | | Material | mg/g |
|---|---|---|---|
| 1. | Drug | S-citalopram Oxalate micronized* | 256.0 |
| | layering | Sugar Spheres, USP | 672.8 |
| | | Hydroxypropyl Cellulose, NF | 51.2 |
| | | Purified Water, USP | 0 |
| 2. | Top | Hydroxypropyl Cellulose | 20.0 |
| | coating | Purified Water, USP | 0 |
| | | Total MR beads | 1000.0 |

*1.28 mg of oxalate salt equiv. to 1 mg of base.

The dissolution data (0.1N HCl, basket 100 rpm) for the exemplary immediate release bead formulations with 100 mg/g escitalopram and 200 mg/g escitalopram described above is provided in Table 6.

TABLE 6
Dissolution Data for 100 mg/g and
200 mg/g escitalopram IR beads

Batch number

| | BN0001355 | BN0001370 |
|---|---|---|
| Time, hr | IR Beads, 100 mg/g | IR Beads, 200 mg/g |
| 0 | 0 | 0 |
| 0.5 | 98 | 99 |
| 1.0 | 98 | 99 |
| 2.0 | 98 | 99 |
| Assay | 95.9% | 96.4% |

Thus, using the exemplary IR bead formulations, various dosages forms of escitalopram may be prepared by filling the capsule with the appropriate amount of the desired bead. For example, 4 mg, 8 mg and 16 mg escitalopram capsules may be prepared as shown in Table 7.

TABLE 7
Composition of capsules having 4, 8 and 16 mg escitalopram
prepared using 100 mg/g and 200 mg/g IR Beads

| | Type of IR Beads | | | | | |
|---|---|---|---|---|---|---|
| | 100 mg/g drug loading | | | 200 mg/g Drug loading | | |
| Dose | 4 mg | 8 mg | 16 mg | 4 mg | 8 mg | 16 mg |
| Amount of IR | 40 | 80 | 160 | 20 | 40 | 80 |
| Beads, mg | | | | | | |
| Gelatin Capsules, | 48 | 48 | 48 | 48 | 48 | 48 |
| Size 3, mg | | | | | | |
| Totals weight mg | 88 | 128 | 208 | 68 | 88 | 128 |

Modified release beads may be prepared by coating immediate release beads (see, e.g., Example 2) with polymer. A coating solution is prepared by mixing an ethyl cellulose polymer dispersion (Surelease®, Colorcon, West Point, Pa.) with water using a stirrer for at least 15 minutes or until fully dissolved. An example of a formulation of the coating solution is shown in Table 8.

TABLE 8
Exemplary coating solution composition.

| Material | g/kg |
|---|---|
| Ethyl cellulose (Surelease)* | 600 |
| Purified Water** | 400 |
| Total | 1 kg |

*Contains 25% w/w solids.
**Purified water is removed during the process.

The immediate release beads are coated with the coating solution to different weight gains (e.g., 3, 6.5, 9%) to achieve different modified release dissolution profiles. The immediate release beads may be coated using a fluidized bed coater (such as GPCG3 manufactured by Glatt Fluid Air, Ramsey, N.J.) and using the following process parameters (for batch size=1.0-3.0 Kg): Product temperature=38-45° C.; Air flow=200-350 m³/h; Spray rate=15-22 gm/min; Atomization pressure=1.0-2.0 bar. One skilled in art will recognize that different concentrations of polymer and amounts applied will also be acceptable. The coated beads are dried at an inlet temperature of 45-55° C. in the fluid bed for up to 1 hour.

An optional seal coat of about 2% to about 5% w/w may then be applied to the beads. The seal coating solution (Table 9) is prepared by mixing hydroxypropyl methylcellulose (Opadry Colorcon) with water using a stirrer until it is fully dissolved. The beads are coated with the seal coating solution using a fluidized bed coater (such as GPCG3 manufactured by Glatt fluid Air, Ramsey, N.J.) and using the following process parameters (for batch size=1.0-3.0 Kg): Product temperature=40-50° C.; Air flow=200-350 m³/h; Spray rate=9-42 gm/min; Atomization pressure=1.5-2.0 bar. The coated beads are dried at an inlet temperature of 45-55° C. in the fluid bed for up to 1 hour. Alternatively, the coated beads can be dried in oven at about 40-50° C. for up to 72 hours.

TABLE 9
Optional seal coating solution

| Material | g/kg |
|---|---|
| Hydroxypropyl Cellulose (Opadry) | 70 |
| Purified Water* | 930 |
| Total | 1 kg |

*Purified water is removed during the process.

Three different modified release beads have been prepared: Slow release (MR bead I); intermediate release (MR bead II); and fast release (MR bead III). The composition of each of the exemplary bead formulations are shown in Tables 10-12, respectively.

TABLE 10
Formulation for 91.5 mg/g escitalopram
MR Beads slow release (MR bead I)

| Process stage | | Material | mg/g |
|---|---|---|---|
| 1. | Drug | S-citalopram Oxalate micronized | 117.2 |
| | layering | Sugar Spheres, USP | 756.4 |
| | | Hydroxypropyl Cellulose, NF | 41.7 |
| | | Purified Water, USP | 0 |
| 2. | MR | Take above Escitalopram IR Beads, | 915.3 |
| | coating | 100 mg/g | |
| | | Surelease* | 238.0 |
| | | Purified Water, USP | 0 |
| 3. | Top | Opadry Clear (YS-1-7006) | 25.2 |
| | coating | Purified Water, USP | 0 |
| | | Total MR beads | 1000.0 |

*Contains 25% w/w solids.

TABLE 11
Formulation for, 92.8 mg/g escitalopram MR
Beads intermediate release (MR bead II)

| Process stage | | Material | mg/g |
|---|---|---|---|
| 1. | Drug | escitalopram Oxalate micronized | 118.8 |
| | layering | Sugar Spheres, USP | 767.0 |
| | | Hydroxypropyl Cellulose, NF | 42.4 |
| | | Purified Water, USP | 0 |
| 2. | MR | Take above Escitalopram IR Beads, | 928.1 |
| | coating | 100 mg/g | |
| | | Surelease* | 185.6 |
| | | Purified Water, USP | 0 |
| 3. | Top | Hydroxypropyl Cellulose (Opadry) | 25.5 |
| | coating | Purified Water, USP | 0 |
| | | Total MR beads | 1000.0 |

*Contains 25% w/w solids.

TABLE 12
Formulation for, 94.6 mg/g escitalopram
MR Beads fast release (MR bead III)

| Process stage | | Material | mg/g |
|---|---|---|---|
| 1. | Drug | S-citalopram Oxalate micronized | 121.0 |
| | layering | Sugar Spheres, USP | 781.4 |
| | | Hydroxypropyl Cellulose, NF | 43.1 |
| | | Purified Water, USP | 0 |
| 2. | MR | Take above Escitalopram IR Beads, | 945.6 |
| | coating | 100 mg/g | |
| | | Surelease Clear | 113.5 |
| | | Purified Water, USP | 0 |
| 3. | Top | Hydroxypropyl Cellulose (Opadry) | 26.0 |
| | coating | Purified Water, USP | 0 |
| | | Total MR beads | 1000.0 |

*Contains 25% w/w solids.

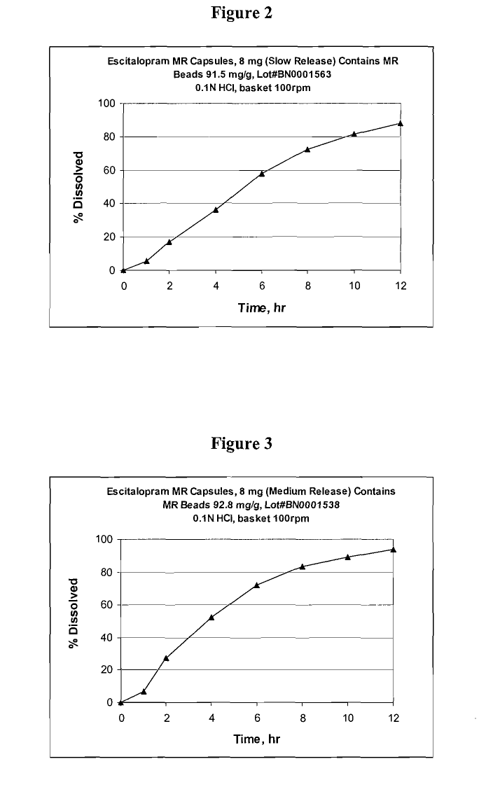

FIGS. 2-4 show the dissolution profile for the slow release (MR bead I); intermediate release (MR bead II); and fast release (MR bead III), respectively. Table 13 shows the pharmacokinetic parameters (Cmax, AUC and Tmax) for immediate release escitalopram (currently marketed Lexapro®), slow release (MR bead I), intermediate release (MR bead II) and fast release (MR bead III).

TABLE 13
Mean ± SD Pharmacokinetic Parameters of IR and MR formulations (normalized for dose of 8 mg)

| | | | | | MR II vs | MR I vs | MR III vs |
|---|---|---|---|---|---|---|---|
| PK | | MR I | MR II | MR III | IR | IR | IR |
| Parameters | IR | Slow | Intermediate | Fast | 90% CI | 90% CI | 90% CI |
| Cₘₐₓ (ng/mL) | 9.22 ± 1.63 | 5.07 ± 2.72 | 5.07 ± 1.09 | 8.20 ± 1.75 | 46-56 | 49-60 | 80-98 |
| | | (51%)* | (54%)* | (88%)* | | | |
| AUC₀₋ₜ | 271 ± 129 | 202 ± 92 | 221 ± 114 | 263 ± 119 | 68-80 | 73-86 | 91-106 |
| (hr* ng/mL) | | (73%)* | (79%)* | (97%)* | | | |
| AUC_{0-inf} | 321 ± 143 | 273 ± 89 | 284 ± 117 | 317 ± 130 | 83-94 | 84-96 | 94-106 |
| (hr* ng/mL) | | (88%)* | (90%)* | (99%)* | | | |
| Tmax Hour | 5 ± 3 | 12.5 ± 5 | 11.7 ± 5 | 6.8 ± 1 | | | |

( )*: (%) Ratio (Test/Reference) of geometric mean
CI: confidence interval

### Example 4

### Modified Release Tablets

A modified release escitalopram tablet may be prepared as a matrix formulation. Three different modified release tablets have been prepared: Slow release (MR tablet I); intermediate release (MR tablet II); and fast release (MR tablet III). The composition of each of the exemplary tablet formulations are shown in Tables 14-16, respectively.

TABLE 14
Formulation for escitalopram modified release
tablets, slow release (MR tablet I)

| | | | Weight |
|---|---|---|---|
| Material | Function | Percentage % | (mg/tablet) |
| S-citalopram Oxalate* | API | 8.5% | 10.2 |
| Hydroxypropylmethyl- | Polymer | 60.0% | 72.0 |
| cellulose (Synchron KF) | | | |
| ProSolv SMCC 90 | filler | 23.0% | 27.6 |
| Talc, USP | glidant | 5.0% | 6.0 |
| Magnesium Stearate, NF | lubricant | 1.0% | 1.2 |
| Opadry Clear (YS-1-7006) | Coating | 2.5% | 3.0 |
| Total | — | 100% | 120.0 |

*1.28 mg of oxalate salt equiv. to 1 mg of base.

TABLE 15
Formulation for escitalopram modified release
tablets, intermediate release (MR tablet II)

| | | | Polymer (Filler) | |
|---|---|---|---|---|
| | | | Synchron 40% | Synchron 40% |
| | | | (ProSolv) | (Lactose) |
| | | | Lot# | |
| | | | RD-1318-22A | RD-1318-22C |
| | Function | Percentage % | mg/tablet | mg/tablet |
| S-citalopram Oxalate* | API | 8.5% | 10.2 | 10.2 |
| Hydroxypropylmethylcellulose | Polymer | 48.0% | 48.0 | 48.0 |
| (Synchron KF) | | | | |
| ProSolv SMCC 90 | filler | 0-53.4% | 53.4 | 0 |
| Lactose monohydrate, NF | filler | 0-54.6% | 0 | 54.6 |
| Talc, USP | glidant | 6.0% | 6.0 | 6.0 |
| Colloidal Silicon Dioxide, NF | glidant | 0-1.2% | 1.2 | 0 |
| Magnesium Stearate, NF | lubricant | 1.2% | 1.2 | 1.2 |
| Total | — | 100% | 120 | 120 |

*1.28 mg of oxalate salt equiv. to 1 mg of base.

TABLE 16
Formulation for escitalopram modified release tablets, fast release (MR tablet III)

| | | | PEO | |
|---|---|---|---|---|
| | | | 20 | 25 |
| | | | Synchron | |
| | | | 20 | 20 |
| | | | Lot# | |
| | | | RD-1318-58C | RD-1318-58E |
| | Function | Percentage % | mg/tablet | mg/tablet |
| S-citalopram Oxalate | API | 8.5% | 10.2 | 10.2 |
| Hydroxypropylmethylcellulose | Polymer | 0-20.0% | 24.0 | 24.0 |
| (Synchron KF) | | | | |
| Polyethylene Oxide, NF (MW 200,000) | Polymer | 20.0-40.0% | 24.0 | 30.0 |
| Lactose monohydrate, NF | Filler | QS | 54.6 | 48.6 |
| Talc, USP | Glidant | 5.0% | 6.0 | 6.0 |
| Magnesium Stearate, NF | Lubricant | 1.0% | 1.2 | 1.2 |
| Total | — | 100% | 120 | 120 |

*1.28 mg of oxalate salt equiv. to 1 mg of base.

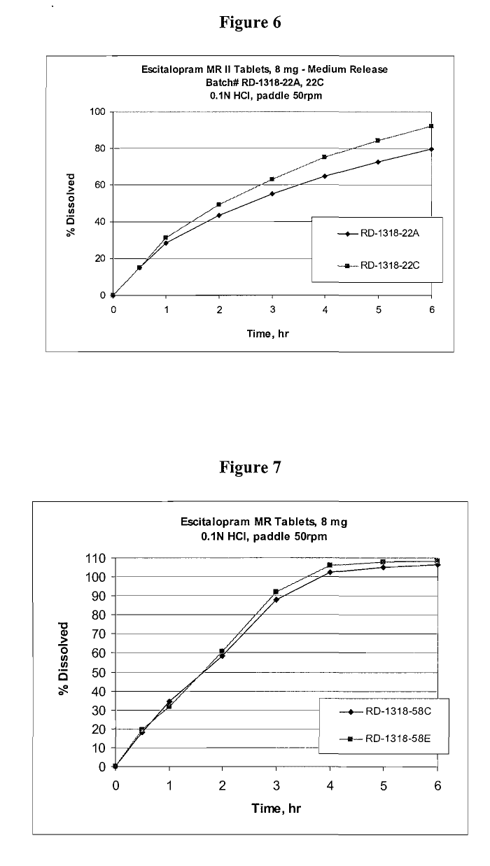

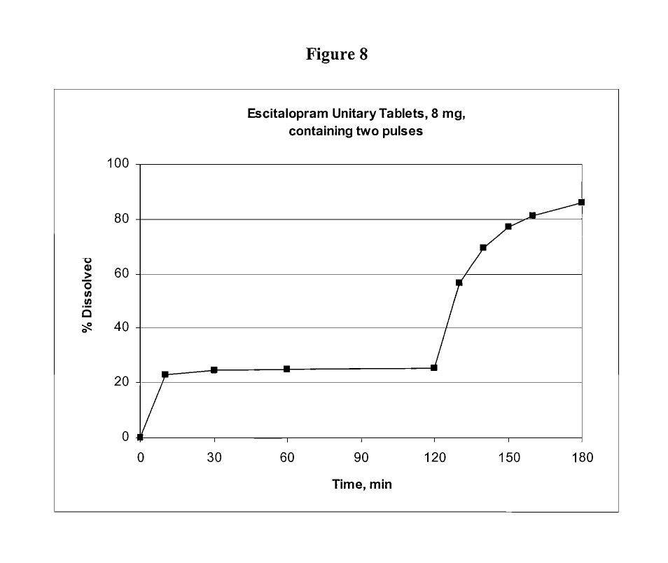

FIGS. 5-7 shows the dissolution profile for the slow release (MR tablet I); intermediate release (MR tablet II); and fast release (MR tablet III), respectively. The slow release tablet (MR tablet I) was prepared using HPMC and the release rate can be optimized by controlling the amount of HPMC. The intermediate release tablet (MR tablet II) may be formulated using either a single polymer or a more than one polymer in combination, e.g., Polyethylene Oxide (POLYOX) and/or HPMC (Synchron). In general, increasing the molecular weight of the polymer will lead to an increase in the gel strength, decreasing the diffusion of the drug. Thus, an increase in molecule weight while maintaining the polymer concentration constant will reduce the release rates. In addition, filler, e.g., water-insoluble ProSolv or water-soluble lactose, can be used to modulate dissolution rate. Other excipients include Talc as a glidant and Magnesium Stearate as a lubricant. The fast release tablet (MR tablet II) formulation may also use either a single polymer or multiple polymers in combination. Other excipients that may be used include lactose as a filler, Talc as a glidant and Magnesium Stearate as a lubricant.

Modified release escitalopram tablets may also be prepared by coating an immediate release tablet (see, e.g., Example 1) with a delayed release polymer (e.g., Eudragit (Acryl-EZE®)). One skilled in art will recognize, upon reading the present disclosure that different polymers, e.g., cellulose acetate phthalate, can also be used to achieve various dissolution profiles and that depending upon the polymer the release rate can be modulated from pH 5.5 to pH 7.4.

### Example 5

### Unitary Tablet Containing Two Pulses

A unitary tablet with two pulses may be prepared as follows: an immediate release tablet that includes 5 mg escitalopram (see Example 1) may be coated with Eudragit (Acryl-EZE®) to provide a delayed release tablet. The tablet may then be coated with escitalopram using HPMC (Opadry) binder. The tablet may optionally be further coated with a seal coat HPMC (Opadry). An exemplary unitary tablet formulation is shown in Table 17.

TABLE 17
unitary tablet formulation of escitalopram

| | mg/100 mg tablet |
|---|---|
| Escitalopram tablets (see Example 1) | 5 |
| Eudragit (Acryl-EZE ®) | 21 |
| Escitalopram | 3 |
| HPMC (Opadry) | 3 |
| HPMC (Opadry) | 4 |

The coating may be performed in a suitable perforated pan coater (Accela Coater, Thomas Engineering, III). The immediate release pulse is released rapidly, e.g., in less than 60 minutes in 0.1 N HCl. The remainder of the drug is substantially released at pH>5.5 (i.e., about 2 hours after exposure).

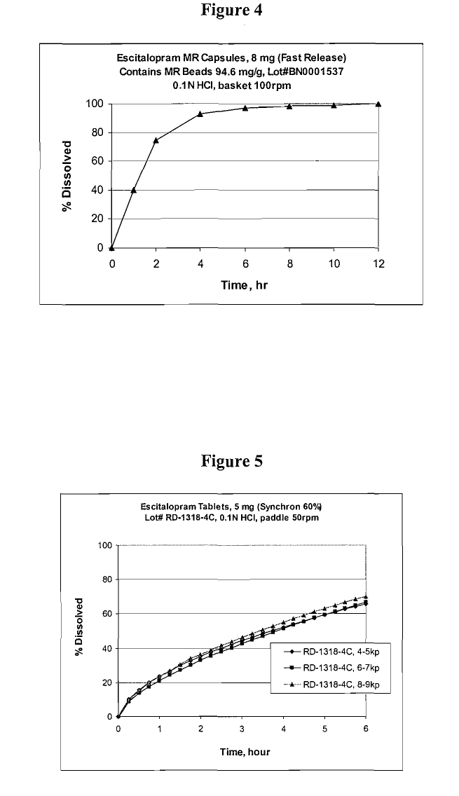

FIG. 4 shows the dissolution profile of an exemplary unitary tablet with two pulses (i.e., a pulsatile tablet). The dissolution profile shows that the first pulse is released rapidly followed by the second pulse release upon exposure to pH>5.5. One skilled in art will recognize that different polymers can also be used to generate pulsatile release formulations.

### Example 6

### Capsule Formulations

Capsule formulations may be prepared to achieve an oral dosage form with optimized pharmacokinetic parameters. For example, it is desirable to provide an oral dosage form that provides the maximum amount of escitalopram to an individual, measured as the area under the plasma concentration-time curve (AUC₀₋ₜ, and AUC_{0-∞}), while minimizing the maximum plasma concentration (Cₘₐₓ) that is produced. Moreover, it is desirable to provide a maximum plasma concentration (Cₘₐₓ) after a specific amount, i.e., control Tₘₐₓ. Capsule compositions that include multiple beads or tablets with the same or different dissolution and/or pharmacokinetic parameters may be used to provide a desired oral dosage form

For example, a capsule comprised of 50% immediate release beads (see Example 2) and 50% intermediate release beads (MR bead II) (see Example 3) may be prepared. For example, 8 mg dosage forms of escitalopram may be prepared by adding 40 mg of the immediate release beads and 43 mg of the intermediate release beads (MR bead II). Thus, dosage forms with varied amounts of escitalopram beads may be prepared (e.g., 2 mg, 4 mg, 5 mg, 8 mg, 10 mg, 15 mg, 16 mg, 20 mg and 30 mg), as shown in Table 18.

TABLE 18
dosage forms of escitalopram comprised of 50% immediate release
beads and 50% intermediate release beads (MR bead II)

| | Dose | | | | | | | | |
|---|---|---|---|---|---|---|---|---|---|
| | 2 mg | 4 mg | 5 mg | 8 mg | 10 mg | 15 mg | 16 mg | 20 mg | 30 mg |
| Amount of IR beads | 10 | 20 | 25 | 40 | 50 | 75 | 80 | 100 | 150 |
| (100 mg/g) | | | | | | | | | |
| Amount of MR II beads | 11 | 22 | 27 | 43 | 54 | 81 | 86 | 108 | 162 |
| (92.8 mg/g) | | | | | | | | | |
| Gelatin Capsules, mg* | 48 | 48 | 48 | 48 | 48 | 76 | 76 | 76 | 96 |
| Totals weight mg | 69 | 90 | 100 | 131 | 152 | 232 | 242 | 284 | 408 |

*Size-3 capsule weight 48 mg, Size-1 capsule weight 76 mg, Size-0 capsule weight 96 mg

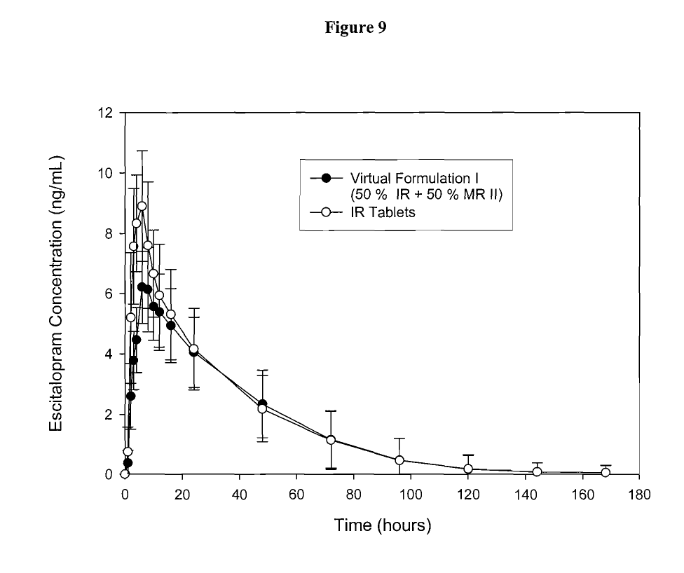

The calculated pharmacokinetic profile for an 8 mg dosage form of escitalopram comprised of 50% IR beads (see Example 2) and 50% MR bead II (see Example 3) are shown in FIG. 9. The pharmacokinetic profile was calculated using the data shown in FIG. 1. FIG. 9 also shows the calculated pharmacokinetic profile for an 8 mg immediate release tablet of escitalopram (see Example 1). Table 19 shows the pharmacokinetic parameters for the 50% IR bead and 50% MR bead II dosage form and immediate release tablets of escitalopram.

TABLE 19
(Mean ± SD) Plasma Pharmacokinetic Parameters
of 50% IR beads and 50% MR bead II (calculated) and
8 mg immediate release tablets of escitalopram

| | A | | (%) Ratio | |
|---|---|---|---|---|
| | (50% IR + | | (A/B) of | |
| | 62.5% MR II) | B | geometric | |
| PK Parameters | (calculated) | (100% IR) | mean | 90% CI |
| Cₘₐₓ (ng/mL) | 6.4 ± 1.2 | 9.2 ± 1.6 | 69 | 66.14-72.02 |
| AUC₀₋ₜ (hr · | 248 ± 119 | 271 ± 129 | 91 | 87.43-93.91 |
| ng/mL) | | | | |
| AUC_{0-∞} (hr · | 295 ± 128 | 321 ± 143 | 92 | 89.42-95.53 |
| ng/mL) | | | | |

A: Single oral dose of 8 mg of virtual formulation I (50% IR + 50% MR II)
B: Single oral dose of 8 mg IR formulation calculated using 10 mg IR tablet data

In another example, a capsule comprised of 37.5% immediate release beads (see Example 2) and 62.5% intermediate release beads (MR bead II) (see Example 3) may be prepared. For example, 8 mg dosage forms of escitalopram may be prepared by adding 30 mg of the immediate release beads and 54 mg of the intermediate release beads (MR bead II). Thus, dosage forms with varied amounts of escitalopram may be prepared (e.g., 2 mg, 4 mg, 5 mg, 8 mg, 10 mg, 15 mg, 16 mg, 20 mg and 30 mg), as shown in Table 20.

TABLE 20
dosage forms of escitalopram comprised of 37.5% immediate release
beads and 62.5% intermediate release beads (MR bead II)

| | Dose | | | | | | | | |
|---|---|---|---|---|---|---|---|---|---|
| | 2 mg | 4 mg | 5 mg | 8 mg | 10 mg | 15 mg | 16 mg | 20 mg | 30 mg |
| Amount of IR beads | 7.5 | 15 | 19 | 30 | 38 | 56 | 60 | 75 | 113 |
| (100 mg/g) | | | | | | | | | |
| Amount of MR II | 13.5 | 27 | 34 | 54 | 67 | 101 | 108 | 135 | 203 |
| beads (92.8 mg/g) | | | | | | | | | |
| Gelatin Capsules, mg* | 48 | 48 | 48 | 48 | 48 | 76 | 76 | 76 | 96 |
| Totals weight mg | 69 | 90 | 101 | 132 | 153 | 233 | 244 | 286 | 412 |

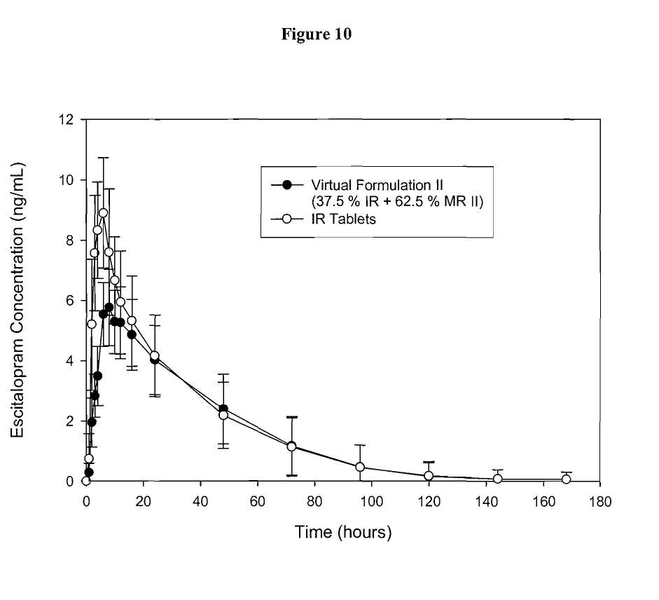

The calculated pharmacokinetic profile for an 8 mg dosage form of escitalopram comprised of 37.5% IR beads (see Example 2) and 62.5% MR bead II (see Example 3) are shown in FIG. 10. The pharmacokinetic profile was calculated using the data shown in FIG. 1. FIG. 10 also shows the calculated pharmacokinetic profile for an 8 mg immediate release tablet of escitalopram (see Example 1).Table 21 shows the pharmacokinetic parameters for the 37.5% IR bead and 62.5% MR bead II dosage form and immediate release tablets of escitalopram.

TABLE 21
(Mean ± SD) Plasma Pharmacokinetic Parameters
of 37.5% IR beads and 62.5% MR bead II (calculated)
and 8 mg immediate release tablets of escitalopram

| | A | | (%) Ratio | |
|---|---|---|---|---|
| | (37.5% IR + | | (A/B) of | |
| | 62.5% MR II) | B | geometric | |
| PK Parameters | (calculated) | (100% IR) | mean | 90% CI |
| Cₘₐₓ (ng/mL) | 5.9 ± 1.2 | 9.2 ± 1.6 | 64 | 60.28-66.51 |
| AUC₀₋ₜ (hr · | 242 ± 118 | 271 ± 129 | 88 | 84.22-91.98 |
| ng/mL) | | | | |
| AUC_{0-∞} (hr · | 289 ± 125 | 321 ± 143 | 91 | 86.97-94.72 |
| ng/mL) | | | | |

A: Single oral dose of 8 mg of virtue formulation II (37.5% IR + 62.5% MR II)
B: Single oral dose of 8 mg IR formulation calculated using 10 mg IR tablet data

In another example, a capsule comprised of 50% immediate release beads (see Example 2) and 50% slow release beads (MR bead I) (see Example 3) may be prepared. For example, 8 mg dosage forms of escitalopram may be prepared by adding 40 mg of the immediate release beads and 44 mg of the slow release beads (MR bead I). Thus, dosage forms with varied amounts of escitalopram may be prepared (e.g., 2 mg, 4 mg, 5 mg, 8 mg, 10 mg, 15 mg, 16 mg, 20 mg and 30 mg), as shown in Table 22.

TABLE 22
dosage forms of escitalopram comprised of 50% immediate
release beads and 50% slow release beads (MR bead I)

| | Dose | | | | | | | | |
|---|---|---|---|---|---|---|---|---|---|
| | 2 mg | 4 mg | 5 mg | 8 mg | 10 mg | 15 mg | 16 mg | 20 mg | 30 mg |
| Amount of IR beads | 10 | 20 | 25 | 40 | 50 | 75 | 80 | 100 | 150 |
| (100 mg/g) | | | | | | | | | |
| Amount of MR II | 11 | 22 | 27 | 44 | 55 | 82 | 87 | 109 | 164 |
| beads (92.8 mg/g) | | | | | | | | | |
| Gelatin Capsules, mg* | 48 | 48 | 48 | 48 | 48 | 76 | 76 | 76 | 96 |
| Totals weight mg | 69 | 90 | 100 | 132 | 153 | 233 | 243 | 285 | 410 |

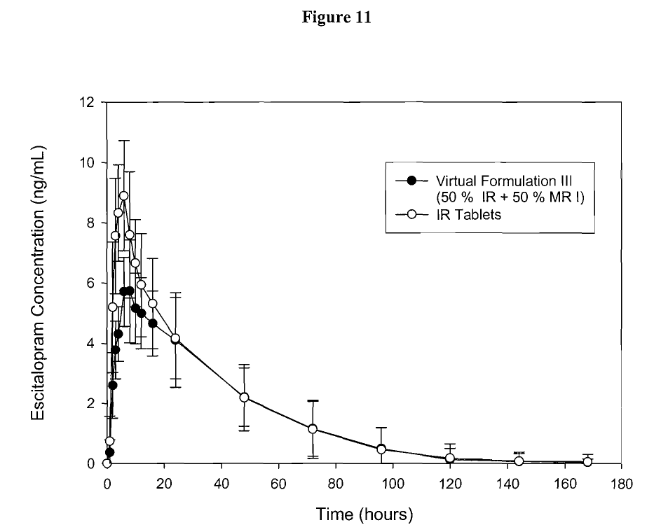

The calculated pharmacokinetic profile for an 8 mg dosage form of escitalopram comprised of 50% IR beads (See Example 2) and 50% MR bead I (see Example 3) are shown in FIG. 11. The pharmacokinetic profile was calculated using the data shown in FIG. 1. FIG. 11 also shows the calculated pharmacokinetic profile for an 8 mg immediate release tablet of escitalopram (see Example 1). Table 23 shows the pharmacokinetic parameters for the 50% IR bead and 50% MR bead I dosage form and immediate release tablets of escitalopram.

TABLE 23
(Mean ± SD) Plasma Pharmacokinetic Parameters
of 50% IR beads and 50% MR bead I (calculated) and
8 mg immediate release tablets of escitalopram

| | A | | (%) Ratio | |
|---|---|---|---|---|
| | (50% IR + | | (A/B) of | |
| | 50% MR I) | B | geometric | |
| PK Parameters | (calculated) | (100% IR) | mean | 90% CI |
| Cₘₐₓ (ng/mL) | 6.3 ± 1.7 | 9.2 ± 1.6 | 67 | 62.39-71.36 |
| AUC₀₋ₜ (hr · | 239 ± 108 | 271 ± 129 | 88 | 84.63-91.51 |
| ng/mL) | | | | |
| AUC_{0-∞} (hr · | 283 ± 107 | 321 ± 143 | 90 | 86.47-93.40 |
| ng/mL) | | | | |

A: Single oral dose of 8 mg of virtue formulation III (50% IR + 50% MR I)
B: Single oral dose of 8 mg IR formulation calculated using 10 mg IR tablet data

In another example, a capsule comprised of 37.5% immediate release beads (see Example 2) and 62.5% slow release beads (MR bead I) (see Example 3) may be prepared. For example, 8 mg dosage forms of escitalopram may be prepared by adding 30 mg of the immediate release beads and 55 mg of the slow release beads (MR bead I). Thus, dosage forms with varied amounts of escitalopram may be prepared (e.g., 2 mg, 4 mg, 5 mg, 8 mg, 10 mg, 15 mg, 16 mg, 20 mg and 30 mg), as shown in Table 24.

TABLE 24
dosage forms of escitalopram comprised of 37.5% immediate
release beads and 62.5% slow release beads (MR bead I)

| | Dose | | | | | | | | |
|---|---|---|---|---|---|---|---|---|---|
| | 2 mg | 4 mg | 5 mg | 8 mg | 10 mg | 15 mg | 16 mg | 20 mg | 30 mg |
| Amount of IR beads | 7.5 | 15 | 19 | 30 | 38 | 56 | 60 | 75 | 114 |
| (100 mg/g) | | | | | | | | | |
| Amount of MR I beads | 17 | 27 | 34 | 55 | 68 | 102 | 109 | 137 | 204 |
| (91.5 mg/g) | | | | | | | | | |
| Gelatin Capsules, mg* | 48 | 48 | 48 | 48 | 48 | 76 | 76 | 76 | 96 |
| Totals weight mg | 72.5 | 90 | 101 | 133 | 154 | 234 | 245 | 288 | 414 |

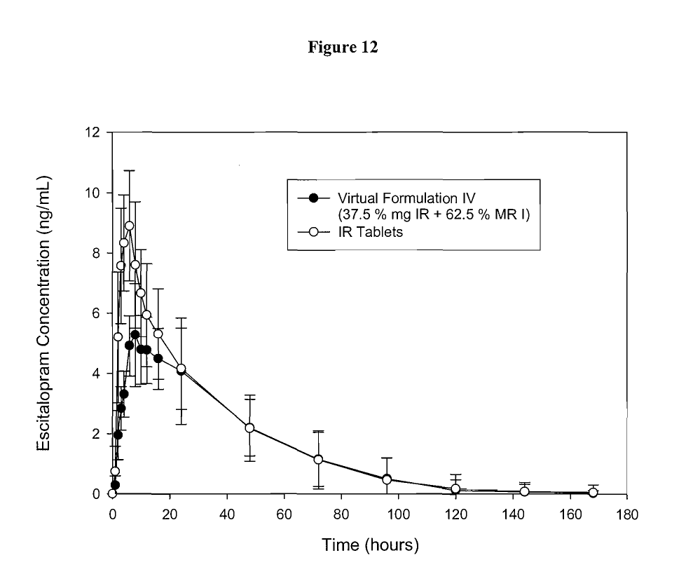

The calculated pharmacokinetic profile for an 8 mg dosage form of escitalopram comprised of 37.5% IR beads (See Example 2) and 62.5% MR bead I (see Example 3) are shown in FIG. 12. The pharmacokinetic profile was calculated using the data shown in FIG. 1. FIG. 12 also shows the calculated pharmacokinetic profile for an 8 mg immediate release tablet of escitalopram (see Example 1). Table 25 shows the pharmacokinetic parameters for the 37.5% IR bead and 62.5% MR bead II dosage form and immediate release tablets of escitalopram.

TABLE 25
(Mean ± SD) Plasma Pharmacokinetic Parameters
of 37.5% IR beads and 62.5% MR bead I (calculated)
and 8 mg immediate release tablets of escitalopram

| | A | | (%) Ratio | |
|---|---|---|---|---|
| | (37.5% IR + | | (A/B) of | |
| | 62.5% MR I) | B | geometric | |
| PK Parameters | (calculated) | (100% IR) | mean | 90% CI |
| Cₘₐₓ (ng/mL) | 5.8 ± 1.9 | 9.2 ± 1.6 | 60 | 55.00-66.07 |
| AUC₀₋ₜ (hr · | 230 ± 104 | 271 ± 129 | 85 | 80.58-88.92 |
| ng/mL) | | | | |
| AUC_{0-∞} (hr · | 277 ± 99 | 321 ± 143 | 88 | 83.93-92.78 |
| ng/mL) | | | | |

A: Single oral dose of 8 mg of virtue formulation IV (37.5% IR + 62.5% MR I)
B: Single oral dose of 8 mg IR Formulation calculated using 10 mg IR tablet data

In other examples, capsules may comprise beads with both an immediate release and a modified release component. The immediate release component of the bead may have the same formulation as an optimized immediate release bead. Similarly, the modified release component may have the same formulation as an optimized modified release bead. Thus, a dosage form with enhanced pharmacokinetic parameters may be prepared. For example, an oral dosage form that provides the maximum amount of escitalopram to an individual, measured as the area under the plasma concentration-time curve (AUC₀₋ₜ, and AUC_{0-∞}), while minimizing the maximum plasma concentration (Cₘₐₓ) that is produced.

The modified release component may be prepared as described in Example 3. For example, the modified release component may be slow release (MR bead I); intermediate release (MR bead II); or fast release (MR bead III). One skilled in the art upon reading the present disclosure will realize that other modified release may be used as the modified release component of the bead. The modified release component may then be layered with an immediate release component. The immediate release component can be prepared using the layering techniques described in Example 2. The immediate release layer may then be further coated with a top coating. Thus, beads comprising a modified release component and an immediate release component can be prepared as shown in Table 26.

TABLE 26
Single bead with a modified release component (based on MR
bead II composition) and an immediate release component. The
ratio of the IR component and the MR component is 50:50

| | | Weight, | MR |
|---|---|---|---|
| | | mg/ | Beads, |
| | Material | capsule | mg/g |
| Modified | escitalopram Oxalate | 5.1 | 104.5 |
| release | micronized** | | |
| component | Sugar Spheres, USP | 33.1 | 674.6 |
| | Hydroxypropyl | 1.8 | 37.2 |
| | Cellulose, NF | | |
| | Surelease*** | 2.0 | 163.2 |
| Immediate | S-citalopram | 5.1 | 104.5 |
| release | Oxalate micronized** | | |
| component | Hydroxypropyl | 1.0 | 20.9 |
| | Cellulose, NF | | |
| Top coating | Opadry Clear | 0.9 | 17.5 |
| | Total MR beads | 49.0 | 1000.0 |

*IR bead manufacturing process has two steps, as shown details in IR beads section.
**1.28 mg of oxalate salt equiv. to 1 mg of base
***Contains 25% w/w solids.

Using the beads described in Table 26 capsules with various dosages of escitalopram (e.g., 2 mg, 4 mg, 5 mg, 8 mg, 10 mg, 15 mg, 16 mg, 20 mg and 30 mg) may be prepared, as shown in Table 27. For example, 8 mg dosage forms of escitalopram may be prepared by filling a capsule with 49 mg of the beads. Table 28 shows the calculated pharmokinetic parameters for the modified release capsules of Table 27. Thus, dosage forms with varied amounts of escitalopram may be prepared.

TABLE 27
Escitalopram dosage forms comprised of the beads of Table 26.

| | Dose | | | | | | | | |
|---|---|---|---|---|---|---|---|---|---|
| | 2 mg | 4 mg | 5 mg | 8 mg | 10 mg | 15 mg | 16 mg | 20 mg | 30 mg |
| Amount of unitary MR Beads, | 12.5 | 25 | 31 | 49 | 61 | 92 | 98 | 123 | 183 |
| 163.3 mg/g | | | | | | | | | |
| Gelatin Capsules, Size 3, mg | 48 | 48 | 48 | 48 | 48 | 48 | 48 | 48 | 76 |
| Totals, mg/capsule | 60.5 | 72 | 79 | 97 | 109 | 140 | 146 | 170 | 259 |

TABLE 28
Calculated pharmokinetic parameters for modified
release capsules of escitalopram

| | Dosage (mg) | | | | | | | | | |
|---|---|---|---|---|---|---|---|---|---|---|
| | 2 | 4 | 5 | 8 | 10 | 15 | 16 | 20 | 25 | 30 |
| Cmax | 1.6 | 3.2 | 4.0 | 6.4 | 8.0 | 12.0 | 12.8 | 16.0 | 20.0 | 24.0 |
| AUC 24 | 62.0 | 124.0 | 155.0 | 248.0 | 310.0 | 465.0 | 496.0 | 620.0 | 775.0 | 930.0 |
| AUC a | 73.8 | 147.5 | 184.4 | 295.0 | 368.8 | 553.1 | 590.0 | 737.5 | 921.9 | 1106.3 |
| Tmax | 6 | 6 | 6 | 6 | 6 | 6 | 6 | 6 | 6 | 6 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description. Patents, patent applications, publications, product descriptions, and protocols are cited throughout this application, the disclosures of which are incorporated herein by reference in their entireties for all purposes.

What is claimed is:

1. An oral dosage form comprising from about 2 mg to about 30 mg escitalopram or a pharmaceutically acceptable salt thereof, wherein the dosage form provides an in vivo plasma profile comprising:
a mean Tmax of more than about 6 hours;
a mean Cmax of less than about 30 ng/ml; and
a mean AUC_{0-∞} of more than about 60 ng h/ml.

2. The oral dosage form according to claim 1, wherein the dosage form comprises from about 5 mg to about 20 mg escitalopram or a pharmaceutically acceptable salt thereof.

3. The oral dosage form according to claim 1, wherein the dosage form comprises from about 4 mg to about 16 mg escitalopram or a pharmaceutically acceptable salt thereof.

4. The oral dosage form according to claim 1, wherein the dosage form provides an in vivo plasma profile comprising a mean Tmax of more than about 8 hours.

5. The oral dosage form according to claim 1, wherein the dosage form provides an in vivo plasma profile comprising a mean Cmax of less than about 10.0 ng/ml.

6. The oral dosage form according to claim 1, wherein the dosage form provides an in vivo plasma profile comprising a mean Cmax of less than about 5.0 ng/ml.

7. The oral dosage form according to claim 1, wherein the dosage form provides an in vivo plasma profile comprising a mean AUC_{0-∞} of more than about 120 ng h/ml.

8. The oral dosage form according to claim 1, wherein the dosage form provides an in vivo plasma profile comprising a mean AUC_{0-∞} of more than about 150 ng h/ml.

9. The oral dosage form according to claim 1, wherein the dosage form provides an in vivo plasma profile comprising a mean AUC_{0-∞} of more than about 300 ng h/ml.

10. The oral dosage form according to claim 1, wherein the dosage form is selected from the group consisting of a tablet, a capsule, a bead and combinations thereof.

11. The oral dosage form according to claim 1, wherein the dosage form comprises about 2 mg escitalopram or a pharmaceutically acceptable salt thereof and provides an in vivo plasma profile comprising:
a mean Tmax of more than about 6 hours;
a mean Cmax of less than about 2 ng/ml; and
a mean AUC_{0-∞} of more than about 60 ng h/ml.

12. The oral dosage form according to claim 1, wherein the dosage form comprises about 4 mg escitalopram or a pharmaceutically acceptable salt thereof and provides an in vivo plasma profile comprising:
a mean Tmax of more than about 6 hours;
a mean Cmax of less than about 4 ng/ml; and
a mean AUC_{0-∞} of more than about 120 ng h/ml.

13. The oral dosage form according to claim 1, wherein the dosage form comprises about 5 mg escitalopram or a pharmaceutically acceptable salt thereof and provides an in vivo plasma profile comprising:
a mean Tmax of more than about 6 hours;
a mean Cmax of less than about 5 ng/ml; and
a mean AUC_{0-∞} of more than about 150 ng h/ml.

14. The oral dosage form according to claim 1, wherein the dosage form comprises about 10 mg escitalopram or a pharmaceutically acceptable salt thereof and provides an in vivo plasma profile comprising:
a mean Tmax of more than about 6 hours;
a mean Cmax of less than about 10 ng/ml; and
a mean AUC_{0-∞} of more than about 300 ng h/ml.

15. The oral dosage form according to claim 1, wherein the dosage form comprises about 20 mg escitalopram or a pharmaceutically acceptable salt thereof and provides an in vivo plasma profile comprising:
a mean Tmax of more than about 6 hours;
a mean Cmax of less than about 20 ng/ml; and
a mean AUC_{0-∞} of more than about 600 ng h/ml.

16. The oral dosage form according to claim 1, wherein the dosage form comprises about 30 mg escitalopram or a pharmaceutically acceptable salt thereof and provides an in vivo plasma profile comprising:
a mean Tmax of more than about 6 hours;
a mean Cmax of less than about 30 ng/ml; and
a mean AUC_{0-∞} of more than about 900 ng h/ml.

17. An oral dosage form comprising about 2 to about 30 mg escitalopram or a pharmaceutically acceptable salt thereof wherein the dosage form has a dissolution rate of the active ingredient of about 70% to about 80% within about 4 hours to about 24 hours and provides an in vivo plasma profile comprising a mean Cₘₐₓ of less than about 30 ng/ml.

18. The oral dosage form according to claim 17, wherein the dosage form provides an in vivo plasma profile comprising a mean AUC_{0-∞} of more than about 120 ng h/ml.

19. The oral dosage form according to claim 17, wherein the dosage form provides an in vivo plasma profile comprising a mean AUC_{0-∞} of more than about 150 ng h/ml.

20. The oral dosage form according to claim 17, wherein the dosage form comprises about 2 mg escitalopram or a pharmaceutically acceptable salt thereof and an in vivo plasma profile comprising a mean AUC_{0-∞} of more than about 60 ng h/ml and a mean Cₘₐₓ of less than about 2 ng/ml.

21. The oral dosage form according to claim 17, wherein the dosage form comprises about 4 mg escitalopram or a pharmaceutically acceptable salt thereof and an in vivo plasma profile comprising a mean AUC_{0-∞} of more than about 120 ng h/ml and a mean Cₘₐₓ of less than about 4 ng/ml.

22. The oral dosage form according to claim 17, wherein the dosage form comprises about 5 mg escitalopram or a pharmaceutically acceptable salt thereof and an in vivo plasma profile comprising a mean AUC_{0-∞} of more than about 150 ng h/ml and a mean Cₘₐₓ of less than about 5 ng/ml.

23. The oral dosage form according to claim 17, wherein the dosage form comprises about 10 mg escitalopram or a pharmaceutically acceptable salt thereof and an in vivo plasma profile comprising a mean AUC_{0-∞} of more than about 300 ng h/ml and a mean Cₘₐₓ of less than about 10 ng/ml.

24. The oral dosage form according to claim 17, wherein the dosage form comprises about 20 mg escitalopram or a pharmaceutically acceptable salt thereof and an in vivo plasma profile comprising a mean AUC_{0-∞} of more than about 600 ng h/ml and a mean Cₘₐₓ of less than about 18 ng/ml.

25. The oral dosage form according to claim 17, wherein the dosage form comprises about 30 mg escitalopram or a pharmaceutically acceptable salt thereof and an in vivo plasma profile comprising a mean AUC_{0-∞} of more than about 900 ng h/ml and a mean Cₘₐₓ of less than about 30 ng/ml.

26. A composite dosage form comprising an immediate release component and a modified release component,
wherein the immediate release component comprises a first active ingredient comprising about 2 to about 30 mg escitalopram or a pharmaceutically acceptable salt thereof, wherein about 80% of the first active ingredient dissolves within about the first 4 hours following entry of the dosage form into a use environment; and
wherein the modified release component comprises a second active ingredient comprising about 2 to about 30 mg escitalopram or a pharmaceutically acceptable salt thereof, wherein about 70% to about 80% of the second active ingredient dissolves within about 4 hours to about 24 hours following entry of the dosage form into the use environment.

27. The composite dosage form of claim 26, wherein the immediate release component comprises a bead, a tablet or a particle comprising escitalopram or a pharmaceutically acceptable salt thereof.

28. The composite dosage form of claim 26, wherein the modified release component comprises a bead, a tablet or a particle comprising escitalopram or a pharmaceutically acceptable salt thereof.

29. An oral dosage form comprising a plurality of beads, each bead comprising
a core having a diameter from about 1 μm to about 1000 μm;
an active ingredient comprising about 2 to about 30 mg escitalopram or a pharmaceutically acceptable salt thereof; and
a modified release coating,
wherein the oral dosage form has a dissolution rate of the active ingredient of about 70% to about 80% within about 4 hours to about 24 hours; and wherein the dosage form provides an in vivo plasma profile comprising an in vivo plasma profile comprising a mean Cₘₐₓ of less than about 30 ng/ml.

30. The oral dosage form according to claim 29, wherein the dosage form provides an in vivo plasma profile comprising a mean AUC_{0-∞} of more than about 120 ng h/ml.

31. The oral dosage form according to claim 29, wherein the release modifying polymer is selected from the group consisting of ethylcellulose (Surelease®), methacrylate (Eudragit®), methacrylic acid copolymer type C (Acryl-eze®), and mixtures thereof.

32. The oral dosage form according to claim 29, further comprising a top coating coated on the release modifying polymer layer.

33. The oral dosage form according to claim 32, wherein the top coating is selected from the group consisting of HPMC (Opadry®), HPC, Eudragit® RL, Eudragit® E100, Eudragit® E 12.5, Eudragit® E PO, Eudragit® NE, and mixtures thereof.

34. An oral dosage form comprising a plurality of beads, each bead comprising
a first drug component comprising a core comprising about 500 to about 800 mg sugar per gram of bead, about 30 mg to about 300 mg escitalopram or a pharmaceutically acceptable salt thereof per gram of bead and a first polymer comprising about 20 mg to about 60 mg per gram of bead;
a modified release coating comprising about 50 to about 300 mg per gram of bead;
a second drug component comprising about 50 mg to about 150 mg escitalopram or a pharmaceutically acceptable salt thereof per gram of bead and a second polymer comprising about 5 mg to about 50 mg per gram of bead; and
optionally, a top coating comprising about 5 mg to about 25 mg per gram of bead,
wherein the oral dosage form has a dissolution rate of the active ingredient of about 70% to about 80% within about 4 hours to about 24 hours.

35. The oral dosage form of claim 34, wherein the first drug component comprises a core comprising about 675 mg sugar per gram of bead, about 105 mg escitalopram oxalate per gram of bead and the first polymer comprises about 37 mg hydroxypropyl cellulose per gram of bead;
the modified release coating comprises about 163 mg surelease per gram of bead;
the second drug component comprises about 105 mg escitalopram oxalate per gram of bead and the second polymer comprises about 21 mg hydroxypropyl cellulose per gram of bead; and
the top coating comprises about 17 mg opadry clear per gram of bead.

36. A drug capsule comprising about 2 mg escitalopram or a pharmaceutically acceptable salt thereof wherein the capsule comprises the oral dosage form of claim 34 and provides an in vivo plasma profile comprising: a mean Tmax of more than about 6 hours; a mean Cmax of less than about 2 ng/ml; and a mean AUC_{0-∞} of more than about 60 ng h/ml.

37. A drug capsule comprising about 4 mg escitalopram or a pharmaceutically acceptable salt thereof wherein the capsule comprises the oral dosage form of claim 34 and provides an in vivo plasma profile comprising: a mean Tmax of more than about 6 hours; a mean Cmax of less than about 4 ng/ml; and a mean AUC_{0-∞} of more than about 120 ng h/ml.

38. A drug capsule comprising about 5 mg escitalopram or a pharmaceutically acceptable salt thereof wherein the capsule comprises the oral dosage form of claim 34 and provides an in vivo plasma profile comprising: a mean Tmax of more than about 6 hours; a mean Cmax of less than about 5 ng/ml; and a mean AUC_{0-∞} of more than about 150 ng h/ml.

39. A drug capsule comprising about 10 mg escitalopram or a pharmaceutically acceptable salt thereof wherein the capsule comprises the oral dosage form of claim 34 and provides an in vivo plasma profile comprising a mean Tmax of more than about 6 hours; a mean Cmax of less than about 10 ng/ml; and a mean AUC_{0-∞} of more than about 300 ng h/ml.

40. A drug capsule comprising about 20 mg escitalopram or a pharmaceutically acceptable salt thereof wherein the capsule comprises the oral dosage form of claim 34 and provides an in vivo plasma profile comprising a mean Tmax of more than about 6 hours; a mean Cmax of less than about 20 ng/ml; and a mean AUC_{0-∞} of more than about 600 ng h/ml.

41. A drug capsule comprising about 30 mg escitalopram or a pharmaceutically acceptable salt thereof wherein the capsule comprises the oral dosage form of claim 34 and provides an in vivo plasma profile comprising a mean Tmax of more than about 6 hours; a mean Cmax of less than about 30 ng/ml; and a mean AUC_{0-∞} of more than about 900 ng h/ml.

42. A method of treating a central nervous system disorder comprising administering to a patient in need thereof a therapeutically effective amount of the dosage form according to claim 1.

43. The method of claim 42, wherein the central nervous system disorder is selected from the group consisting of mood disorders, anxiety disorders, premenstrual dysphoric disorder, premenstrual syndrome, neurotic disorders, acute stress disorder, eating disorders, phobias, dysthymia, cognitive disorders, impulse control disorders, attention deficit hyperactivity disorder and drug abuse.

44. The method of claim 42, wherein the mood disorder is major depressive disorder.

45. The method of claim 42, wherein the anxiety disorder is selected from the group consisting of generalized anxiety disorder, social anxiety disorder, post traumatic stress disorder, obsessive compulsive disorder and panic disorder.

46. The method of claim 42, wherein the eating disorder is selected from the group consisting of binge eating, bulimia, anorexia and obesity.
